(19) United States
(12) Patent Application Publication
(10) Pub. No.: US 2018/0140637 A1
(43) Pub. Date: May 24, 2018

(54) EPIDERMAL NEURAL CREST STEM CELLS AS A SOURCE OF SCHWANN CELLS

(71) Applicant: Maya Sieber-Blum, Sante Fe, NM (US)

(72) Inventor: Maya Sieber-Blum, Sante Fe, NM (US)

(21) Appl. No.: 15/574,754

(22) PCT Filed: May 16, 2016
(86) PCT No.: PCT/US16/32735

§ 371 Date: Nov. 16, 2017

Related U.S. Application Data
(60) Provisional application No. 62/163,562, filed on May 19, 2015.

(51) Int. Cl. A61K 35/30; A61K 35/36; A61K 35/545; C12N 5/0797

(52) U.S. Cl. CPC A61K 35/30; A61K 35/36; A61K 35/545; C12N 5/0623; C12N 2500/38; C12N 2501/135; C12N 2501/115; C12N 2501/237; C12N 2500/44; C12N 2501/13

(57) ABSTRACT

The application describes compositions, kits, and methods for generating Schwann cells from epidermal neural crest stem cells (EPI-NCSC). Such EPI-NCSC can be obtained from the bulge of hair follicles.

This application claims the benefit of priority to U.S. Provisional Patent Application Ser. No. 62/163,562 entitled “Epidermal Neural Crest Stem Cells as a Source of Schwann Cells,” filed May 19, 2015, the disclosure of which is incorporated herein by reference in its entirety.

### BACKGROUND

Schwann cells are neural crest-derived glial cells that support axons of peripheral nerves. Injuries to peripheral nerves are common; they can be due to neuropathies, traumatic injury, tumour-related surgery, or repetitive compression (Pfister et al, 2011; Griffin et al 2014; Evans 2001), Overall, peripheral nerve injury carries a high cost to health care systems (Rosberg et al, 2005). After peripheral nerve injury, the proximal stump of the nerve is capable of regeneration and reinnervation. Surgical repair for minor nerve reconstruction involves direct end-to-end anastomosis (Griffin et al, 2014). In significant nerve injuries nerve repair requires bridging the gap and the introduction of Schwann cells, together with Schwann cell-derived growth factors and an extracellular matrix for guiding axonal extension and nerve regeneration (Shirosaki et al. 2014). Axons regenerate along aligned Schwann cells called bands of Btingner, which form a permissive environment for axon regeneration (Allodi et al, 2012). Scaffolds and tubes are used as guidance materials for nerve grafts. Currently, autografts from sensory nerves are typically used to repair large peripheral nerve damage. Autografts have several disadvantages, however, including damage to the donor nerve caused by the biopsy. Schwann cells have also proven useful to treat spinal cord injuries (see e.g., Williams and Bunge, 2012; Bunge, 2008). Overall, the availability of large numbers of human Schwann cells for autologous use, and for disease modelling and drug discovery is highly desirable.

### SUMMARY

The invention relates to Schwann cells that can be derived rapidly and in a straightforward way, without the need for genetic manipulation, from epidermal neural crest stem cells (EPI-NCSC) present in the bulge of hair follicles. These Schwann cells are useful for cell-based therapies, for treatment of various diseases, for disease modelling and for drug discovery. The Schwann cells generated as described herein have advantageous properties. For example, the neural crest is the biologically most pertinent tissue to generate Schwann cells. EPI-NCSC are readily accessible, for example, via punch biopsy from hairy skin. In addition, no purification of EPI-NCSC needed for generation of Schwann cells. Highly pure populations of Schwann cells can be generated using the compositions and methods described herein. Hence, little or no purification of EPI-NCSC derived Schwann cells is needed. The methods described herein do not involve genetic manipulation. Hence, the Schwann cells described herein would not have insertions or genetic modifications that can arise when genetically modified cells are employed. For example, some types of induced pluripotent stem cells can have inserted expression cassettes or vectors that encode heterologous pluripotency factors. If Schwann cells are generated from such induced pluripotent stern cells (iPSC), the iPSC-derived Schwann cells would have mutations that would not be present in the EPI-NCSC derived Schwann cells described herein. Millions of Schwann cells can be obtained within relatively short period of time using the methods and compositions described herein. In addition, human, mouse and canine EPI-NCSC do not form tumors in vivo, Hence, even if small numbers of EPI-NCSC are present in the Schwann cells, those EPI-NCSC would not cause cancer.

Unlike methods that have previously been employed, the methods described herein generate Schwann cells from epidermal neural crest stem cells (EPI-NCSC) that can be obtained from the bulge of hair follicles. The epidermal neural crest stein cells are pre-treated with sonic hedgehog (SHH) and a WNT signaling activator, which increases SOX10 expression in the cells. Such treatment increases KROX20 expression as differentiation proceeds (KROX20 is a gene that is specific for myelinating Schwann cells). Also, methods that have previously been employed, during differentiation the cells are treated with an RARγ agonist for an extended period of time (e.g., for more than 5 days). TGFβ1 inhibitors are also employed in the methods described herein to inhibit neuron differentiation, and thereby promote Schwann cell differentiation.

### DESCRIPTION OF THE FIGURES

FIG. 1 is a schematic diagram of the process for treatment of epidermal neural crest stein cells to generate Schwann cells. The epidermal neural crest stem cells were pre-treated with human recombinant sonic hedgehog (rhSHH) and CHIR99021 for 24 hours in expansion (XP) medium (described hereinbelow; see also Clewes et al. 2011)) to enhance SOX10 expression (Days −3 to Day −1). Cells were then sub-cultured on Day −1 (arrow) into XP medium as indicated and allowed to recover for 24 hours. At Day 0 of differentiation (Day 0), the culture medium was changed to Alpha-modified MEM, SB431542 was added, and the cells were treated with β-mercaptoethanol for 24 hours. Subsequently, at Day 1, retinoic acid was added. On Day 3, the differentiation factors FG-F2, PDGF-BB, Neuregulin and forskolin were first added (third differentiation medium). All six components were added fresh every 48 hours with each culture medium exchange.

FIG. 2A-2G shows images of cellular morphology before and during differentiation. FIG. 2A shows images of cells at Day −3 of differentiation, illustrating that the cells had a stellate morphology typical of neural crest cells. FIG. 2B shows that at Day 0, after SW' and CHIR99021 treatment, cell morphology had not changed noticeably. FIG. 2C shows that by day 4, cells had continued to proliferate and started to change morphology. FIG. 2D illustrates that proliferation continued and cells became elongated by Day 9. FIG. 2E shows that the morphology of cells was maintained in culture for 14 days. FIG. 2F shows that the cellular morphology was maintained in prolonged culture for 25 days. FIG. 2G shows an expanded view of the cellular morphology after culture for 25 days.

FIG. 3A-3F show images of cells at day zero (D0) of differentiation subjected to indirect immunocytochemistry. FIG. 3A shows images of untreated cells (D0) immunostained with anti-SOX10 antibodies. As shown. SOX10 was expressed in all cells, albeit only about half of cells showed nuclear localisation of SOX10 immunoreactivity. FIG. 3B shows images of the same cells (D0 of differentiation) immunostained with anti-p75NTR. (NGFR) antibodies. FIG. 3C shows merged images of the same untreated cells (0 days) shown in FIG. 3A and 3B stained with anti-SOX10 antibodies (red in the original), anti-p75NTR antibodies (green in the original), and DAPI (blue in the original). FIG. 3D shows images of the same area of untreated cells (0 days) as shown in FIG. 3A, immunostained with anti-KROX20 antibodies. FIG. 3E shows images of the same untreated cells (0 days) immunostained with anti-P0 antibodies. FIG. 3F shows merged images of the same cells stained with anti-KROX20 (red in original), anti-P0 antibodies (green in original), and DAPI (blue in original). KROX20 was expressed in all cells, albeit mostly in the cytoplasm; P0 was not expressed at detectable levels.

FIG. 4A-4F are images of cells treated pursuant to the methods described herein for 4 days and then subjected to immunocytochemical staining with various fluorescently labelled antibodies. FIG. 4A shows images of cells after 4 days of differentiation (D4) immunostained with anti-SOX10 antibodies. As shown, SOX10 immunoreactivity in the nucleus was detected in most cells; the remaining cells had cytoplasmic SOX10 immunoreactivity. FIG. 4B shows images of the same cells shown in FIG. 4A and treated as described herein for 4 days, then immunostained with anti-p75NTR (NGFR) antibodies. All cells were p75NTR immunopositive. FIG. 4C shows merged images of the same cells treated as described herein for 4 days and stained with anti-SOX10 antibodies (red in the original), anti-p75NTR antibodies (green in the original), and DAPI (blue in the original). FIG. 4D shows images of the same area of cells as shown in FIG. 4A, treated as described herein for 4 days and immunostained with anti-KROX20 antibodies. KROX20 was detected in all cells with predominantly nuclear localisation. FIG. 4E shows images of the same cells shown in FIG. 4A treated as described herein for 4 days and immunostained with anti-P0 antibodies. All cells showed P0 immunoreactivity at very low levels. FIG. 4F shows merged images of the same cells treated as described herein for 4 days and stained with anti-KROX20 (red in the original), anti-P0 antibodies (green in the original), and DAPI (blue in the original).

FIG 5A-5F are images of cells treated pursuant to the methods described herein for 9 days and then subjected to immunocytochemical analysis using the indicated antibodies. FIG. 5A shows images of cells after 9 days treatment, then immunostained with anti-SOX10 antibodies. As shown, virtually all cells showed intense SOX10 nuclear immunoreactivity; remaining cells were SOX10 immunoreactive in the cytoplasm. FIG. SB shows images of the same cells treated as described herein for 9 days, then immunostained with anti-p75NTR (NGFR) antibodies. All cells expressed p75NTR. FIG. 5C shows merged images of the same cells treated as described herein for 9 days and stained with anti-SOX10 antibodies (red in original), anti-p75NTR antibodies (green in original), and DAPI (blue in original). FIG. 5D shows images of the same area of cells as shown in FIG. 5A, treated as described herein for 9 days and immunostained with anti-KROX20 antibodies. All cells showed strong KROX20 immunoreactivity mostly with nuclear localisation. FIG. 5E shows images of the same cells treated as described herein for 9 days and immunostained with anti-P0 antibodies. Weak P0 immunoreactivity was detected in same cells as shown in FIG. 5B; immunoreactivity was at low detectable levels. Inset: P0 immunoreactivity at higher magnification. FIG. 5F shows merged images of the same cells treated as described herein for 9 days and stained with anti-KROX20 (red in original), anti-P0 antibodies (green in original), and DAPI (blue in original).

FIG. 6A-6F are images of cells treated pursuant to the methods described herein for 14 days and then subjected to indirect immunocytochemistry. FIG. 6A shows images of cells after 14 days treatment, then immunostained with anti-SOX10 antibodies. As shown, strong nuclear SOX10 immunoreactivity was detected in virtually all cells. FIG. 6B shows images of the same cells treated as described herein for 14 days, then immunostained with anti-p75NTR (NGFR) antibodies. All cells expressed p75NTR. FIG. 6C shows merged images of the same cells treated as described herein for 14 days and stained with anti-SOX10 antibodies (red in original), anti-p75NTR antibodies (green in original), and DAPI (blue in original). FIG. 6D shows images of the same area of cells as shown in FIG. 6A, treated as described herein for 14 days and immunostained with anti-KROX20 antibodies. KROX20 immunoreactivity was detected in all cells, mostly in the nucleus. FIG. 6E shows images of the same cells treated as described herein for 14 days and immunostained with anti-P0 antibodies. Weak P0 immunoreactivity was detected in all cells in same area as shown in FIG. 6D; the inset shows an area of larger magnification. FIG. 6F shows merged images of the same cells treated as described herein for 14 days and stained with anti-KROX20 (red in original), anti-P0 antibodies (green in original), and DAPI (blue in original). Bar, 50 μm.

FIG. 7A-7D illustrate the close interaction of hEPI-NCSC derived Schwann cells with neurites from rodent dorsal root ganglion (DRG) in confocal micrographs. Cultures were fixed at 11 days of co-culture with dissociated DRG neurons from adult ‘green mouse’ that emit enhanced green fluorescence (EGFP). FIG. 7A shows images of P0 (P-zero) expression (red fluorescence in original) in hEPI-NCSC derived Schwann cells merged with detection of human nuclei using antibodies (pale blue fluorescence in original). Four human Schwann cell nuclei and associated cells are visible. In addition there is bead-like P0 immunofluorescence visible above Schwann cells. FIG. 7B shows merged images of P0 (red in original) immunoreactivity of Schwann cells with anti-human nuclear antibodies (pale blue in original) and green EGFP fluorescence of DRG neurites in the same area as FIG. 7A, The bead-like P-zero positive structures correspond with the neuronal processes. FIG. 7C shows the boxed area shown in FIG. 7B. Slice thickness, 0.72 μm. FIG. 7D is an orthogonal reconstruction of the area marked in FIG. 7C with an arrow and FIG. 7D shows the close interaction between Schwann cell process and neurite. FIG. 7A and 7C, calibration bars 50 μm.

FIG. 8A-8H illustrate the close interaction of hEPI-NCSC derived Schwann cells with neurites from rodent DRG (E15.5 rat embryo DRG explant) in confocal micrographs. Cultures were fixed at day 21 of co-culture with whole DRG explants from rat embryos. FIG. 8A shows P0 expression as red fluorescence in the original. The fluorescence of the Schwann cell bodies is visible in an aligned orientation. In addition, bead-like structures are visible in filamentous arrangement. FIG. 8B shows neurofilaments (NF) in the same area corresponding to the image in FIG. 8A (where neurofilament fluorescence is shown as green fluorescence). FIG. 8C shows a higher magnification of the boxed area in FIG. 8A. FIG. 8D shows a higher magnification image of the boxed area in FIG. 8B. The orientation of neurites corresponds to orientation of Schwann cells in FIG. 8A. FIG. 8E shows merged images of P-zero expression (red in original) and anti-human nuclear antibody staining (pale blue in original), illustrating that all Schwann cells are of human origin. FIG. 8F shows images of P-zero expression (red in original), anti-human nuclear antibody staining (anti-Hu; pale blue in original), neurofilament expression (green in original) and DAPI nuclear staining (dark blue in original) illustrating that all cells are human derived Schwann cells. FIG. 8G shows images of a higher magnification of the boxed area in FIG. 8E. FIG. 8H shows an image of a larger magnification of the boxed area in FIG. 8F. In FIGS. 8C, 8D, 8G, and 8H the arrowheads of the arrows identify P-zero and neurofilament double-positive structures and point to same locations in each image.

FIG. 9A-9E illustrate the close interaction of hEPI-NCSC derived Schwann cells with neurites from rodent DRG as observed by electron microscopy. The ultrastructure was somewhat compromised due to a long lag time between fixation and processing for electron microscopy. FIG. 9A shows a Schwann cell (with nucleus; nu) attached to the substratum on well bottom (wb). In the cytoplasm mitochondria (dark rounded) and endoplasmic reticulum (elongated double-track structures) are visible. The cell morphology is consistent with Schwann cell morphology. FIG. 9B shows a bundle of neurites in cross section (boxed area) enclosed by processes of two cells, one from the left with visible nucleus (nu) and a process from a cell to the right of the image. FIG. 9C shows a higher magnification image of the boxed area in FIG. 9B. The cytoplasm (cp) of the cell to the left shows mitochondria (m) and the double track lines typical of rough endoplasmic reticulum (rER). Individual neurites (neu) in the cluster of neurites are visible in cross-section. Processes in proximity of the cluster of neurites are marked by arrows; the process originating from the cell to the right is marked by a bracket. FIG. 9D shows a cluster of neurites (neu; to the left) with three neurites in close membrane proximity with a cell to the right, the cytoplasm (cp) of which is visible; wb, well bottom. Neurites are in cross-section with microtubules (dark structures) detectable. FIG. 9E shows a higher magnification image of the boxed area in FIG. 9D. Three neurites have made close contact with a cell to the right (five asterisks); wb, well bottom. Bars; FIG. 9A: 50 μm, FIG. 9B: 5 μm, FIG. 9C, 9D, 9E: 500 nm.

FIG. 10A-10L show (cytoplasmic) SOX10, (reduced) p75NTR, (nuclear) KROX20 and (increased) P0 immunofluorescence on differentiation day 19 (FIG. 10A-10F) and differentiation day 30 (FIG. 10G-10L) of hEPI-NCSC derived Schwann cells generated as described herein. FIG. 10A illustrates cytoplasmic SOX10 expression (red in original) in hEPI-NCSC derived Schwann cells at day 19 of incubation as described herein. FIG. 10B illustrates reduced p75NTR expression (green in original) in hEPI-NCSC derived Schwann cells at day 19 of incubation as described herein. FIG. 10C illustrates merged cytoplasmic SOX10, reduced p75NTR expression, and DAPI nuclear staining (blue in original) in hEPI-NCSC derived Schwann cells at day 19 of incubation as described herein. FIG. 10D illustrates nuclear KROX20 expression (red in original) in hEPI-NCSC derived Schwann cells at day 19 of incubation as described herein. FIG. 10E illustrates increased P0 expression (green in original) in hEPI-NCSC derived Schwann cells at clay 19 of incubation as described herein. FIG. 10F illustrates merged nuclear KROX20, increased P0 expression, and DAPI nuclear staining (blue in original) in hEPI-NCSC derived Schwann cells at day 19 of incubation as described herein. FIG. 10G illustrates cytoplasmic SOX10 expression (red in original) in hEPI-NCSC derived Schwann cells at day 30 of incubation as described herein. FIG. 10H illustrates reduced p75NTR expression (green in original) in hEPI-NCSC derived Schwann cells at day 30 of incubation as described herein. FIG. 10I illustrates merged cytoplasmic SOX10, reduced p75NTR expression, and DAPI nuclear staining (blue in original) in hEPI-NCSC derived Schwann cells at day 30 of incubation as described herein. FIG. 10J illustrates nuclear KROX20 expression (red in original) in hEPI-NCSC derived Schwann cells at day 30 of incubation as described herein. FIG. 10K illustrates increased P0 expression (green in original) in hEPI-NCSC derived Schwann cells at day 30 of incubation as described herein. FIG. 10L illustrates merged nuclear KROX20increased P0 expression, and DAPI nuclear staining (blue in original) in hEPI-NCSC derived Schwann cells at day 30 of incubation as described herein.

FIG. 11A-11F shows images illustrating expression of myelin basic protein (MBP), glial fibrillary acidic protein (GFAP), and S100 by hEPI-NCSC on differentiation day zero (D0) and at differentiation day 9 (D9) during incubation as described herein. FIG. 11A illustrates expression of myelin basic protein (MBP; green in the original), and DAPI nuclear staining (blue in the original) of hEPI-NCSC before incubation as described herein (day 0). FIG. 11B illustrates expression of myelin basic protein (MBP), and DAPI nuclear staining of hEPI-NCSC at day 9 of incubation as described herein. FIG. 11C illustrates expression of glial fibrillary acidic protein (GFAP; green in the original), and DAPI nuclear staining (blue in the original) of hEPI-NCSC before incubation as described herein (day 0). FIG. 11D illustrates expression of glial fibrillary acidic protein (GFAP), and DAPI nuclear staining of hEPI-NCSC at day 9 of incubation as described herein. FIG. 11E illustrates expression of S100 (green in the original), and DAPI nuclear staining (blue in the original) of hEPI-NCSC before incubation as described herein (day 0). FIG. 11F illustrates expression of S100, and DAPI nuclear staining of hEPI-NCSC at day 9 of incubation as described herein.

FIG. 12A-12F shows images illustrating the expression of MBP, GFAP, and S100 by hEPI-NCSC derived Schwann cells on differentiation day 14 (D14) and differentiation day 30 (D30) during incubation as described herein. FIG. 12A illustrates expression of myelin basic protein (MBP; green in the original), and DAPI nuclear staining (blue in the original) by hEPI-NCSC derived Schwann cells after incubation as described herein for 14 days (day 14). FIG. 12B illustrates expression of myelin basic protein (MBP), and DAPI nuclear staining of hEPI-NCSC derived Schwann cells at day 30 of incubation as described herein (day 30). FIG. 12C illustrates expression of glial fibrillary acidic protein (GFAP; green in the original), and DAPI nuclear staining (blue in the original) of hEPI-NCSC derived Schwann cells during incubation as described herein for 14 days. FIG. 12D illustrates expression of glial fibrillary acidic protein (GFAP), and DAPI nuclear staining of hEPI-NCSC derived Schwann cells at day 30 of incubation as described herein. FIG. 12E illustrates expression of S100 (green in the original), and DAPI nuclear staining (blue in the original) of hEPI-NCSC derived Schwann cells during incubation as described herein for 14 days. FIG. 12F illustrates expression of S100, and DAN nuclear staining of hEPI-NCSC derived Schwann cells at day 30 of incubation as described herein.

### DETAILED DESCRIPTION

The methods and compositions described herein provide a readily available source of highly pure mammalian Schwann cells in large numbers. Such Schwann cells are desirable both for use in surgery to repair damage to peripheral nerves and for research purposes. The Schwann cells are derived from epidermal neural crest stem cells (EPI-NCSC) present in the bulge of hair follicles. The EPI-NCSC-derived Schwann cells exhibited characteristic morphology, expressed relevant markers at the protein and RNA levels, and they interacted with axons of rodent dorsal root ganglia in explant culture, demonstrating that the cells are functional Schwann cells.

### Starting Cells

The compositions and methods described herein are used with starting cells that can be epidermal neural crest stem cells (EPI-NCSC) present in the bulge of hair follicles. Such EPI-NCSC can be obtained using methods described in U.S. Pat. No. 8,030,072 and/or Clewes et al., Stem Cell Rev Rep (April 2011), both of which are incorporated herein by reference in their entireties.

No Cd34 transcripts are expressed by mammalian EPI-NCSC starting cells (e.g., as detected in mouse EPI-NCSC cells by gene expression profiling and RT-PCR; Hu. Y F, 2006). The lack of Cd34 expression sets EPI-NCSC apart from the nestin⁺/CD34⁺ follicular cells described by Amoh et al. (Proc. Natl. Acad Sci U S A 102: 5530-34 (2005), as well as from hematopoietic stem cells, and from adipocyte stem cells.

Mammalian epidermal neural crest stem cells are also distinct from, and are not skin-derived precursors (or “SKPs”). For example, the epidermal neural crest stem cell molecular signature does not contain the following SKP markers: Slug, Snail, Twist, Pax3, Sox9, alkaline phosphatase, nexin, versican, nestin, Wnt5a, Sca-1, Shox2, Dermo-1 and CD34 (e.g., as detected in mice). Furthermore, the SKP marker genes including alkaline phosphatase, versican, Akp2, Alx4, Hoxa9, Zic1, Gfra1, Snap91, Cntn1, and Enpp2 are not expressed in EPI-NCSC. See, Hu et al., Stem Cells 24: 2692-2702 (2006), which is incorporated herein by reference in its entirety. However, in some embodiments, skin-derived precursors can be converted into Schwann cells using methods similar to those described herein.

Neural crest contributes to the body organs and tissues, ranking it with the ectoderm, mesoderm, and endoderm as a fourth germ layer (Hall B K., Evol Dev 2: 3-5 (2000)). Neural crest cells are capable of generating neurons, Schwann cells, pigment cells, endocrine cells, odontoblasts, fibroblasts, osteoblasts, chondroblasts, and myofibroblasts.

For example, the anagen hair follicle can be excised at the boundary of dermis and epidermis. Fat cells can be removed mechanically. The hair follicle can be cut to remove the hair bulb. The remaining hair bulb can be cut into two pieces and placed onto CELLstart™ coated culture plate (Invitrogen, Paisley, UK Cat# A10142-01). Coated 24-well or 35 mm plates can be employed. Such excisions are fast and fairly simple to perform. For instance, one person can easily prepare 75-100 bulge explants in one session. The tissues typically adhere to the substratum within 1 h. The explants can be incubated in a humidified atmosphere at 37° C., 5% CO₂ and 5% O₂. To protect cells against oxidative stress, neural crest stem cells can be cultured at lower oxygen tension. In general, 5% oxygen does not constitute hypoxia for at least some types of EPI-NCSC, as hypoxia is defined as O₂ tension below the normoxic value in a given tissue (Jezek et al. The International Journal of Biochemistry & Cell Biology, 42(5): 604-622 (2010)). Oxygen tension in hair follicles ranges between 2.5% and 0.1% O₂.

Isolated EPI-NCSC (e.g., human EPI-NCSC) can be cultured as primary explants for 6-8 days, after which they can be sub-cultured as adherent cells at 1×10³ to cells 1×10⁴ cells per 35 mm culture plate (pre-coated with CELLstart™ (Invitrogen) or collagen and fibronectin) under suitable conditions to produce a substantially pure population of EPI-NCSC, which can be readily expanded by subculture. One example of a culture medium that can be employed is NeuroCult XF (Stem Cell Technologies, Grenoble, France Cat# 05761) supplemented with 10 ng/ml rhFGF2 (R&D Systems, Abingdon, UK Cat# 233-FB), 20 ng/ml rhEGF (R&D Systems Cat# 236-EG), 1× ITS+3 (Sigma, Poole, UK Cat# I-2771), 1% (v/v) FBS (HyClone, Thermo Fisher, Cramlington, UK Cat# SH30070.02), 1X GlutaMAX (Invitrogen, Cat# 35050-038), 1× Penicillin/Streptomycin (Sigma Cat# P0781) and 2.5 μg/ml Amphotericin B (Sigma Cat# A2942). The NeuroCult XF medium with these added supplements is referred to herein as XP (expansion) medium. Another example of a culture medium that can be employed is alpha-modified MEM culture medium, ITS+3, chick embryo extract, and members of the following families of growth factors: fibroblast Effowth factors, epidermal growth factors, stem cell factor, neurotrophins.

One human hair follicle can yield, on average, at least three million multipotent stem cells per bulge explant within 28 days. Bulge explants from other mammals can yield different numbers of EPI-NCSC. For example, mouse bulge explains can yield 2.2×10³ cells per explant, within 3-4 days after the onset of emigration from the bulge explant in primary culture. The isolation of the EPI-NCSC from many mammalian hair follicles can be equal or greater than 24×expansion in secondary culture, which equals 52,800 cells per explant within 11-12 days. This typically involves 3-4 days culture as a primary explant plus 8 days in subculture. Moreover, the isolation of the EPI-NCSC ftom mammalian (e.g., mouse) hair follicles is equal or greater than 844,800 cells per mouse within 14-16 days. This is typically 3-4 days in primary explant culture before onset of emigration of cells, plus 3-4 days after the onset of cell emigration and 8 days in subculture.

A substantially pure population of multipotent mammalian non-embryonic EPI-NCSC can be employed in the methods described herein. Such EPI-NCSC can be derived from the epidermal bulge region of a hair follicle. The EPI-NCSC can have a purity level of greater than 70%. Typically 83% EPI-NCSC at the onset of emigration from the hair follicle, are multipotent, are capable of undergoing directed differentiation to give rise to neural crest cells, are highly proliferative in vitro culture, are highly motile, exhibit a high degree of plasticity, are stellate shape in morphology, and are characterized by the positive expression of Sox-10, Nestin (an intermediate filament protein found in cells such as neural precursors) and the expressed neural crest stem cell signature marker genes described in the Examples. See also, U.S. Pat. No. 8,030,072, which is specifically incorporated herein by reference in its entirety.

### Differentiation Methods

The EPI-NCSC can be differentiated into Schwann cells by a method that includes several steps.

The first step can include culturing the EPI-NCSC for at least 1 day, or for at least two days, or for about 1 to about 5 days in a pre-treatment culture medium that contains sonic hedgehog and a WNT signaling activator (e.g., CHIR99021). In some cases, the EPI-NCSC are cultured in a pre-treatment culture medium at day −3 of differentiation. The sonic hedgehog (SHH) and a WNT signaling activator are employed in amounts sufficient to increase nuclear localization of SOX10 expression. Further description of the pre-treatment culture medium is provided hereinbelow. After pre-treatment, the EPI-NCSC generally start to express Schwann cell markers. For example, the EPI-NCSC exhibit expression of KROX20, a gene expressed specifically in myelinating Schwann cells (Allodi I et al, 2012). The EPI-NCSC also express the Schwann cell markers myelin basic protein (MBP), and S100. However, gliat fibrillary acidic protein (GFAP) immunoreactivity is not detectable in EPI-NCSC after pre-treatment.

After about 1 to about 5 days, or after about 1 or 2 days, the EPI-NCSC can be sub-cultured. For example, EPI-NCSC can be sub-cultured at a density of about 10,000-60,000, or about 20,000 to 44,000 cells per 35 mm culture plate. In some cases, the EPI-NCSC are sub-cultured at day −1 of differentiation. The cells can be incubated for about 1 day in a culture medium that does not include sonic hedgehog or a WNT signaling activator. For example, the EPI-NCSC can be incubated in the same type of media as is used for the pre-treatment culture medium without the sonic hedgehog and a WNT signaling activator.

The day after sub-culturing the cells, the EPI-NCSC can be cultured in a first differentiation culture medium containing a first amount of serum, a reducing agent, and an inhibitor of transforming growth factor-beta 1 (TGFβ1 inhibitor). The start date for incubation in the first differentiation culture medium is referred to as day zero (D0) of differentiation. The EPI-NCSC can be cultured in a first differentiation culture medium for at least one day, at least two days, at least three days, or about 1 to 4 days to generate a first cell population.

For example, the reducing agent can be a disulphide bond reducing agent. The reducing agent can be present in the first differentiation culture medium for at least 6 hours, or at least 12 hours, or at least 1 day, or at least 2 days. In some cases, the cells are cultured in the presence of the reducing agent for about 1 day.

The cells can be cultured in the first differentiation culture medium that contains a first amount of serum for at least 1 day or at least two days or at least three days. In some cases, the cells are cultured in the presence of a first amount of serum for about three days.

The TGFβ1 inhibitor can be present in the first differentiation culture medium, and in the subsequently employed culture media, for the remainder of the differentiation process.

Further description of the first differentiation culture medium is provided hereinbelow.

The day after addition of the reducing agent, a RARγ agonist can be added to the culture medium. In some cases, the cells addition of the RARγ agonist occurs at day 1 of differentiation. Hence, the medium containing the RARγ agonist can be removed and the cells can be cultured in a second differentiation culture medium that includes the RARγ agonist, a first amount of serum, and a TGFβ1 inhibitor. If the culture medium is not exchanged (e.g., the RARγ agonist is just added to the existing medium), there may be some reducing agent in the second differentiation culture medium. The cells are incubated in the second differentiation culture medium for at least 1 day, or at least two days, or at least three days. In some cases, the cells are incubated in the second differentiation culture medium for about 2 days.

The RARγ agonist can be present in the second differentiation culture medium and in the culture media used throughout the remainder of the differentiation process.

The same TGFβ1 can be employed in the first and the second differentiation culture media. The TGFβ1 inhibitor is present in the second differentiation culture medium at the same concentration as the first differentiation culture medium.

Further information on the second differentiation culture medium is provided hereinbelow.

After treatment with the second differentiation culture medium the majority of cells express KROX20 and SOX10 in the nucleus. For example, about 65% to 95% of cells express KROX20 and SOX10 in the nucleus after treatment with the second differentiation culture medium. P-zero (P0) expression can be detected at low levels in in the cells after treatment with the second differentiation culture medium (but not in EPI-NCSC). Glial fibrillary acidic protein (GFAP) immunoreactivity is still not detected in the cells after treatment with the second differentiation culture medium.

At about two to four days after the sub-culturing of the EPI-NCSC (e.g., at about day 3 of differentiation), the following can be added to the culture medium: a fibroblast growth factor, a platelet derived growth factor, a neuregulin, and an agent that elevates cAMP levels. These components can be present in the culture medium for the remainder of the differentiation process. Hence, the cells can be incubated in a third differentiation culture media that contains a RARγ agonist, a TGFβ1 inhibitor, a second amount of serum, a fibroblast growth factor, a platelet derived growth factor, a neuregulin, and an agent that elevates cAMP levels. The RARγ agonist, a TGFβ1 inhibitor, a second amount of serum, a fibroblast growth factor, a platelet derived growth factor, a neuregulin, and an agent that elevates cAMP levels are employed for a time and at a concentration that blocks the cells from becoming neurons and instead promotes differentiation of the cells into Schwann cells.

The serum concentration employed in the third differentiation culture medium is generally less than that employed in the second differentiation culture medium, for example, as described hereinbelow. The RARγ agonist and the TGFβ1 inhibitor can be used at the same concentration as in the second differentiation culture medium.

The fibroblast growth factor, a platelet derived growth factor, a neuregulin, and an agent that elevates cAMP levels can be present in a variety of concentrations as described hereinbelow.

The following describes the method employed in the Examples for differentiating EPI-NCSC into Schwann cells. In some cases, the EPI-NCSC can be differentiated into Schwann cells by a method that includes about three days of incubation in a pre-treatment culture medium containing about 500 ng/ml recombinant human sonic hedgehog (C24II) N-terminus (rhSHH) and about 500 nM CHIR99021 for two days. After about two days, the cells can be sub-cultured by detachment, centrifugation, and plating in medium at a density of about 20,000-50,000 cells per 35 mm culture plate and 1,000 cells per poly-D-lysine and laminin-coated 13-mm diameter glass coverslip. The next day (differentiation Day 0), cells can be cultured in a first differentiation culture medium (e.g., Alpha-modified MEM) in the presence of 10% FBS and 1 mM β-mercaptoethanol (βME) for 24 hours, followed by addition of 35 ng/ml all-trans-retinoic acid the next day. Starting at differentiation Day 0, 10 μM SB431542 is added to the culture medium. Hence, on differentiation day 1 and 2, the cells are present in a second differentiation culture medium containing 10% FBS, 35 ng/ml all-trans-retinoic acid, and 10 μM SB431542. At differentiation Day 3, the FBS concentration can be reduced to about 1% FBS. The all-trans retinoic acid and SB431542 can remain in the culture medium throughout the differentiation process at the same concentrations described above (35 ng/ml all-trans-retinoic acid and 10 μM SB431542). At Day 3, 10 ng/ml rhFGF2, 5 ng/ml recombinant human PDGF-BB (rhPDGF-BB), 5 μM forskolin, 200 ng/ml recombinant human neuregulin-1 (rhNRG) can be added to the culture medium, to form a third differentiation culture medium. The third differentiation culture medium is used throughout the remainder of the differentiation process. The culture medium can be exchanged every about 48 hours. These methods are particularly effective for generating human Schwann cells from human EPI-NCSC.

Glial fibrillary acidic protein (GFAP) immunoreactivity is not detected during the early stages of differentiation but it is detectable at low levels after about Day 14 of differentiation. P-zero (P0) immunoreactivity is also not detectable in EPI-NCSC but becomes apparent at low levels by differentiation Days 4-9, increases by Day 14, and remains strongly detectable in the later stages of differentiation after Day 14.

Schwann cell morphology becomes apparent by about 4 to 9 days after differentiation Day 0. Significant numbers of Schwann cells are present in the culture medium after about 9 days, or by about 10 days, or by about 12 days, or by about 14 days after differentiation Day 0.

### Compositions

The EPI-NCSC can be differentiated into Schwann cells in a series of media, each containing a variety of components. Each of the culture media can be supplied as a liquid composition that contains all of the components for facilitating at least one step of EPI-NCSC differentiation into Schwann cells. Alternatively, each of the culture media can be supplied as a liquid medium with some of the components for facilitating EPI-NCSC differentiation into Schwann cells provided separately as liquid concentrates or as dry formulations.

For example, the EPI-NCSC can first be incubated in a pre-treatment culture medium containing Sonic Hedgehog and a a WNT signaling activator. The pre-treatment culture medium can be a medium that supports, nourishes, and promotes proliferation of EPI-NCSC. Media that can be used to support and nourish EPI-NCSC include, for example, a serum-free medium. For example, the culture medium can be an XP medium (made from NeuroCult XF medium from Stem Cell Technologies (Grenoble, France) with supplements such as FGF2, EGF, L-alanyl-L-glutamine dipeptide, one or more antibiotic, one or more anti-fungal, or a combination thereof). Other examples are described in the Examples and in U.S. Pat. No. 8,030,072, which is incorporated herein by reference in its entirety.

To facilitate differentiation of EPI-NCSC into Schwann cells the selected population of EPI-NCSC can be contacted or mixed with a pre-treatment culture medium that contains Sonic Hedgehog and one or more a WNT signaling activators for a time and at a concentration sufficient to facilitate differentiation or re-direction of the cells to the Schwann cell lineage. For example, the sonic hedgehog (SHH) and a WNT signaling activator are employed for a time and in amounts sufficient to increase nuclear SOX10 expression and SOX10 localization in the cells.

The sonic hedgehog can, for example, be present in the pre-treatment culture medium at a concentration of about 50 ng/ml to about 2 mg/ml, or about 100 ng/ml to about 1.5 mg/ml, or about 200 ng/ml to about 1 mg/ml. The WNT signaling activator can be employed in the pretreatment culture medium in a variety of amounts and/or concentrations. For example, the a WNT signaling activator can be employed in the pretreatment culture medium at a concentration of about 1.0 nanomolar to about 2 micromolar in a solution, or about 10.0 nanomolar to about 1.0 micromolar in a solution, or about 100 nanomolar to about 800 nanomolar in a solution, or at about 200 nM to about 700 nM, or at about 500 nanomolar in solution.

After pre-treatment, at least 70% of the cells express SOX10 in the nucleus, or at least 75% of the cells express SOX10 in the nucleus, or at least 80% of the cells express SOX10 in the nucleus, or at least 85% of the cells express SOX10 in the nucleus, or at least 90% of the cells express SOX10 in the nucleus.

The Sonic Hedgehog and a a WNT signaling activator can be provided as a composition, for example, as a liquid concentrate or as a dry mixture that is added to the pretreatment culture medium.

The EPI-NCSC can then be sub-cultured and incubated in a first differentiation culture medium that includes serum, a reducing agent, and a TGFβ1 inhibitor. The base medium for the pretreatment culture medium can be a medium that supports and nourishes EPI-NCSC. One example is a cell culture medium that contains Earle's salts, ribonucleotides, deoxyribonucleosides, and L-glutamine. For example, the medium can be MEM Alpha medium (Corning). Other examples are described in the Examples and in U.S. Pat. No. 8,030,072, which is incorporated herein by reference in its entirety.

To facilitate differentiation of EPI-NCSC into Schwann cells the sub-cultured EPI-NCSC are contacted or mixed with a first differentiation culture medium that contains a reducing agent, a TGFβ1 inhibitor, and a first amount of serum, for a time and at a concentration sufficient to facilitate differentiation or re-direction of the cells to the Schwann cell lineage.

The first amount of serum can be present in the first differentiation culture medium at a concentration of about 5% to about 20%, or about 10%. Various types of serum can be employed. However, in many cases the serum is fetal bovine serum. A reliable source of serum is fetal bovine serum from HyClone.

The reducing agent in the first differentiation culture medium can be a disulphide bond reducing agent. Examples of disulphide bond reducing agents include dithiothreitol (DTT) and β-mercaptoethanol (βME). In some embodiments the disulphide bond reducing agent is β-mercaptoethanol.

The reducing agent can be present in the first differentiation culture medium at a concentration of about 0.1 millimolar to about 10 millimolar, or about 0.2 millimolar to about 5 millimolar, or about 0.5 millimolar to about 1.5 millimolar, or about 1 millimolar.

The TGFβ1 inhibitor can be present in the first differentiation culture medium at a concentration of about 0.5 micromolar to about 30 micromolar in a solution, or about 1.0 micromolar to about 20 micromolar in a solution, or about 3 micromolar to about 15 micromolar in a solution, or about 10 micromolar in a solution.

The serum, the reducing agent, and the TGFβ1 inhibitor can be provided as a composition, for example, as a liquid concentrate. Alternatively, in some instances the reducing agent and the TGFβ1 inhibitor can be provided as a dry mixture that is added to the first differentiation culture medium along with the first amount of serum.

The cells can also be incubated in a second differentiation culture medium that includes serum, a RARγ agonist, and a TGFβ1 inhibitor. The TGFβ1 inhibitor can be present in the second differentiation culture medium at the same concentrations as in the first differentiation culture medium. The serum can be a first amount of serum, which is at the same concentration as in the first differentiation culture medium.

The RARγ agonist can be present in the second differentiation culture medium at a concentration of about 5 ng/ml to about 60 ng/ml, or about 20 ng/ml to about 50 ng/ml, or about 35 ng/ml. Thus, the RARγ agonist can be present essentially throughout the differentiation process, except possibly for the first day when the reducing agent (e.g., γ-mercaptoethanol) is present.

The cells can also be incubated in a third differentiation culture medium that includes a second amount of serum, a RARγ agonist, a TGFβ1 inhibitor, a fibroblast growth factor, a platelet derived growth factor, a neuregulin, and an agent that elevates cAMP levels (e.g., an analog of cAMP).

The serum in the third differentiation culture medium can be fetal bovine serum. The second amount of serum that can be present in the third differentiation culture medium is less than the first amount of serum. For example, the second amount of serum can be about 0.1% to about 4%, or about 0.3 to about 3%, or about 0.5% to about 1.5%, or about 1.0%.

The RARγ agonist and the TGFβ1 inhibitor can be present in the third differentiation culture medium at the same concentrations as in the second differentiation culture media.

There is a family of fibroblast growth factors, with members involved in angiogenesis, wound healing, embryonic development and various endocrine signaling pathways. The FGFs are heparin-binding proteins and interactions with cell-surface-associated heparin sulfate proteoglycans have been shown to be essential for FGF signal transduction. FGFs are key players in the processes of proliferation and differentiation of wide variety of cells and tissues. In general, the third differentiation culture medium contains a basic fibroblast growth factor. For example, the third differentiation culture medium can contain FGF2.

For example, the fibroblast growth factor can be present in the third differentiation culture medium at a concentration of about 1 ng/ml to about 30 ng/ml, or at about 3 ng/ml to about 20 ng/ml, or at about 5 ng/ml to about 15 ng/ml, or about 10 ng/ml.

The platelet derived growth factor (PDGF) is useful for increasing the proliferation, differentiation and/or survival of cells, The PDGF can be any of PDGF-B, PDGF-BB, PDGF-AB, PDGF-C, PDGF-D, PDGF-CC, PDGF-DD, PDGF-BC, PDGF-AC, PDGF-AD, PDGF-BD or a combination thereof. In some cases, the PDGF is PDGF-BB. The PDGF can be present in the third differentiation culture medium at a concentration of about 0.5 ng/ml to about 25 ng/ml, or at about 1 ng/ml to about 15 ng/ml, or at about 2 ng/ml to about 10 ng/ml, or at about 5 ng/ml.

A variety of agents that elevate cAMP levels can be employed in the third differentiation culture medium. The agents that elevate cAMP levels can be agents that raise cAMP levels either by activating adenylyl cyclase or by preventing its degradation, or by inhibiting Gi of the G protein coupled receptor to its GDP (inactive) form. Examples include forskolin, cholera toxin, caffeine, theophylline, bucladesine (dibutyryl-cAMP, db cAMP), pertussis toxin, and cAMP analogs.

The agents that elevate cAMP levels can be present in the third differentiation culture medium at a concentration of about 0.5 μM to about 25 μM, or at about 1 μM to about 15 μM, or at about 2 μM to about 10 μM, or at about 5 μM.

The neuregulin-1 can be present in the third differentiation culture medium at a concentration of about 50 ng/ml to about 500 ng/ml, or at about 100 ng/ml to about 400 ng/ml, or at about 150 ng/ml to about 300 ng/ml, or at about 200 ng/ml.

The third differentiation culture dium of the cultured cells can be exchanged every 48 hours.

### Sonic Hedgehog

Sonic hedgehog can be present in the pretreatment culture medium, and it is useful for increasing SOX10 expression in EPI-NCSC. It is one of three proteins in the mammalian signaling pathway family called hedgehog, the others being desert hedgehog (DHH) and Indian hedgehog (IHH). SHH is the best studied ligand of the hedgehog signaling pathway. It plays a key role in regulating vertebrate organogenesis, such as in the growth of digits on limbs and organization of the brain. Sequence information for human sonic hedgehog is available as Genhank Accession No. Q15465. Further information on Sonic Hedgehog (SHH) is available, for example, in Marigo, Genomics 28: 44-51 (1995) (the contents of which are incorporated herein by reference in its entirety). Recombinant human Sonic Fledgehog is available from R&Dsystems (see website at mdsystems.com/Products1314-SH).

### WNT Signaling Activators

At least one WNT signaling activator can be included in the pre-treatment culture media. Useful inhibitors are those that can increase canonical WNT signaling in EPI-NCSC. Examples of a WNT signaling activators that can be employed in the pre-treatment medium include one or more of the following compounds:
CHIR99021 (6-(2-(4-(2,4-dichlorophenyI)-5-(4-methyl-1H-imidazol-2-yl)pyrimidin-2-ylamino)ethylamino)nicotinonitrile);
1-azakenpaullone (9-Bromo-7,12-dihydro-pyrido[3′,2′:2,3]azepino[4,5-b]-indol-6(5H)-one), BIO ((2′Z,3′E)-6-Bromoindirubin-3′-oxime);
AR-A014418 (N-(4-MethoxybenzyI)-N′-(5-nitro-1,3-thiazol-2-yl)urea);
Indirubin-3′-monoxime;
5-Iodo-indirubin-3′-monoxime;
kenpaullone (9-Bromo-7,12-dihydroindolo-[3,2-d][1]benzazepin-6(5H)-one);
SB-415286 (3-[(3-Chloro-4-hydroxyphenyl)amino]-4-(2-nitro-phenyI)-1H-pyrrole-2,5-dione);
SB-216763 (3-(2,4-Dichlorophenyl)-4-(1-methyl-1H-indol-3-yl)-1H-pyrrole-2,5-dione);
Maybridge SEW00923SC (2-anilino-5-phenyl-1,3,4-oxadiazole);
(Z)-5-(2,3-Memylenedioxyphenyl)imidazolidine-2,4-dione,
TWS119 (3-(6-(3-aminophenyl)-7H-pyrrolo[2,3-d]pyrimidin-4-yloxy)phenol);
CHIR98014 (N2-(2-(4-(2,4-dichlorophenyl)-5-(1H-imidazol-1-yl)pyrimidin-2-ylamino)ethyl)-5-nitropyridine-2,6-diamine);
SB415286 (3-(3-chloro-4-hydroxyphenylamino)-4-(2-nitrophenyl)-1H-pyrrole-2,5-dione);
Tideglusib (also known as NP031112, or NP-12; 1,2,4-Thiadiazolidine-3,5-dione, 2-(1-naphthaleny)-4-(phenylmethyl));
LY2090314 (1H-Pyrrole-2,5-dione, 3-imidazo[1,2-a]pyridin-3-yl-4-[1,2,3,4-tetrahydro-2-(1-piperidinylcarbonyl)pyrrolo[3,2,1-jk][1,4]benzodiazepin-7-yl]);
lithium salt (e.g., LiCl); or
any combination thereof.

The WNT signaling activator can, for example, be CHIR99021, SB216763, TWS119, CHIR98014, Tideglusib, SB415286, LY2090314, or any combination thereof.

In some embodiments, the WNT signaling activator in the pretreatment medium can be CHIR99021, whose structure is shown below.

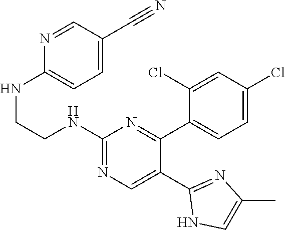 [SMILES: Cc1c[nH]c(-c2cnc(NCCNc3ccc(C#N)cn3)nc2-c2ccc(Cl)cc2Cl)n1]

The WNT signaling activators can also be in the form of a salt or hydrate of any of the foregoing compounds.

In a dry formulation, the WNT signaling activators can be present in amounts useful for addition to 1-100 liters of media. For example, the WNT signaling activators can be present in a dry composition in amounts of about 0.0001 μg to about 1000 mg, or about 0.001 μg to about 100 mg, or about 0.01 mg to about 100 mg can be present in amounts of about 0.1 mg to about 10 mg.

### TGFβI Inhibitors

As illustrated herein use of one or more transforming growth factor-beta (TGF-β) inhibitors can facilitate conversion of EPI-NCSC into Schwann cells.

There are about thirty members of the transforming growth factor-beta (TGF-β) superfamily, including TGF-βs, activin, Nodal, and BMPs. These TGF-β family members elicit their responses through a variety of cell surface receptors that activate Smad protein signaling cascades.

A TGF-beta inhibitor can directly or indirectly, negatively regulate TGF-beta signaling. In some embodiments, one or more TGF-beta inhibitors binds to and reduces the activity of one or more serine/threonine protein kinases selected from the group consisting of ALK5, ALK4, TGF-beta receptor kinase 1 and ALK7, ALK4, ALK5 and ALK7 are all closely related receptors of the TGF-beta superfamily. Desirable TGF-beta inhibitors can bind to and reduce the activity of ALK4, ALK5 (TGF-beta receptor kinase 1) and/or ALK7. In another embodiment, the TGF-beta receptor binds to and reduces the activity of a Smad protein, for example R-SMAD or SMAD1-5 (i.e. SMAD 1, SMAD 2, SMAD 3, SMAD 4 or SMAD 5).

Examples of TGF-B inhibitors include, but are not limited to:
4-[4-(1,3-benzodioxol-5-yl)-5-(2-pyridinyl)-1H-imidazol-2-yl]benzamide (also known as SB 431542 from Tocris Bioscience; a potent and selective inhibitor of TGF-β type I receptor activin receptor-like kinase ALK5 (e.g., with IC₅₀=94 nM), and its relatives ALK4 and ALK7);
3-(6-Methyl-2-pyridinyl)-N-phenyl-4-(4-quinolinyl)-1H-pyrazole-1-carbothioamide (also known as A83-01 from Tocris Bioscience; a selective inhibitor of TGF-β type I receptor ALK5 kinase, type I activin/nodal receptor ALK4 and type I nodal receptor ALK7 (IC50 values can, e.g., be 12, 45 and 7.5 nM respectively); 2-(3-(6-Methylpyridine-2-yl)-1H-pyrazol-4-yl)-1,5-naphthyridine (also known as SJN 2511 from Tocris Bioscience; selective inhibitor of the TGF-β type I receptor ALK5 (IC50 values can, e.g., be 0.004 and 0.023 μM for ALK5 autophosphorylation and ALK5 binding, respectively);
4-[4-(2,3-Dihydro-1,4-benzodioxin-6-yl)-5-(2-pyridinyl)-1H-imidazol-2-yl]benzamide (also known as D 4476 from Tocris Bioscience; a selective inhibitor of casein kinase 1 (CK1) and TGF-β type-1 receptor (ALK5 ) that displays greater than 20-fold selectivity over SAPK2/p38);
4-[3-(2-Pyridinyl)-1H-pyrazol-4-yl]-quinoline (also known as LY 364947 from Tocris Bioscience; a selective inhibitor of TGF-β type-1 receptor (TGF-β R1, TGFR-I, TβR-1, ALK-5) (IC50 values can, e.g., be 59, 400 and 1400 nM for TGR-β RI, TGF-β RII and MLK-7K respectively);
2-(4-(benzo[d][1,3]dioxol-5-yl)-2-tert-butyl-1H-imidazol-5-yl)-6-methylpyridine (also known as SB505124, and available from Selleckchem.com; a selective inhibitor of ALK4 and ALK5 (e.g., with IC50 of 129 nM and 47 nM, respectively);
6-[2-(1,1-Dimethylethyl)-5-(6-methyl-2-pyridinyl)-1H-imidazol -4-yl]quinoxaline (also known as SB 525334 from Sigma-Aldrich; a selective inhibitor of transforming growth factor-β receptor I (ALK5, TGF-βRI), with IC50=14.3 nM, for example);
2-(5-Chloro-2-fluorophenyl)-4-[(4-pyridyl)amino]pteridine (also known as SD 208 from Tocris Bioscience; a potent, orally active ATP-competitive transforming growth factor-β receptor 1 (TGF-βRI) inhibitor, e.g., with IC50=49 nanomolar);
4-(6-(4-(piperazin-1-yl)phenyl)pyrazolo[1,5-a]pyrimidin-3-yl)quinoline (also known as LDN-193189 from Miltenyi Biotec); and
any combination thereof.

The inhibitor that directly or indirectly negatively regulates TGF-beta signaling can, for example, be selected from the group consisting of SB-431542, A83-01, SJN-2511, LY-36494, SB-505124, SB-525334, and SD-208. In some embodiments, inhibitor that directly or indirectly negatively regulates TGF-beta signaling can inhibit ALK4, ALK5 and/or ALK7. For example, the inhibitor that directly or indirectly negatively regulates TGF-beta signaling can be SB431542.

The TGF-beta inhibitor can be employed in the compositions and methods described herein in a variety of amounts and/or concentrations. For example, the TGF-beta inhibitor can be employed at a concentration of about 0.01 micromolar to about 1 millimolar in a solution, or about 0.1 micromolar to about 500 micromolar, or about 1 micromolar to about 50 micromolar. In a dry formulation, the TGF-beta inhibitor can be present in amounts of about 0.1 mg to about 1000 mg, or about 0.5 mg to about 500 mg, or about 1 mg to about 100 mg.

Various methods for determining if a substance is a TGF-beta inhibitor are known. For example, a cellular assay can be used in which cells are stably transfected with a reporter construct comprising the human PAI-1 promoter or Smad binding sites, driving a luciferase reporter gene. Inhibition of luciferase activity relative to control groups can be used as a measure of compound activity (De Gouville et al., Br J Pharmacol. 2005 May; 145(2): 166-177). Mother example is the AlphaScreen® phosphosensor assay for measurement of kinase activity (Drew A E et al., Comparison of 2 Cell-Based Phosphoprotein Assays to Support Screening and Development of an ALK inhibitor J Biomol Screen. 16(2) 164-173, 2011).

In some cases the TGFβ1 inhibitor in the first, second and third differentiation culture media can be SB431542, which has the following structure.

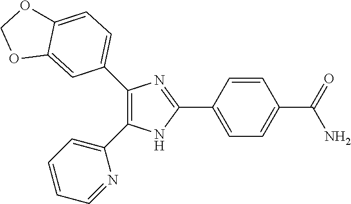 [SMILES: NC(=O)c1ccc(-c2nc(-c3ccc4c(c3)OCO4)c(-c3ccccn3)[nH]2)cc1]

SB431542 facilitated Schwann cell differentiation from EPI-NCSC. SB431542 is a selective and potent inhibitor of ALK-4, -5 and -7. and it inhibits endogenous activin and TGFβ signalling without affecting more divergent BMP signalling utilizing ALK-1, -2, -3 (Laping et al, 2002). Such inhibition may guide stem cells away from the neuronal cell lineage, inhibit TGFβ-related apoptosis early in Schwann cell differentiation, and avoid inhibition of SOX10 and P0 expression by IGFβ signalling (Morgan et al, 1994; Jessen and Mirsky, 2005; Keish, 2006).

The TGFβ1 inhibitors can also be in the form of a salt or hydrate of any of the foregoing compounds.

In a dry formulation, the TGFβ1 inhibitors can be present in amounts useful for addition to 1-100 liters of media. For example, the TGFβ1 inhibitor can be present in a dry composition in amounts of about 0.0001 μg to about 1000 mg, or about 0.001 μg to about 100 mg, or about 001 mg to about 100 mg can be present in amounts of about 0.1 mg to about 10 mg.

### RARγ Agonists

A variety of RARγ agonists can be used in the compositions and methods described herein. For example, RARγ agonists can be present in the second and third differentiation culture media. Examples of RARγ agonists can include:
CD1530 (4-(6-hydroxy-7-tricyclo[3.3.1.13,7]dec-1-yl-2-naphthalenyl)benzoic acid);
CD666 (also known as SureCN12572388, CHEMBL97080, 4-[(E)-3-hydroxy-3-(5,5,8,8-tetramethyl-6,7-dihydronaphthalen-2-yl)prop-1-enyl]benzoic Acid),
NRX204647 (4-((1E,3E)-3-(hydroxyimino)-3-(5,5,8,8-tetratmethyl-5,6,7,8-tetrahydronaphthalen-2-yl)prop-1-en-1-yl)benzoic acid);
retinoic acid;
all-trans retinoic acid (ATRA);
9-cis retinoic acid;
all-trans 3-4 didehydro retinoic acid
4-oxo retinoic acid;
Retinol;
4-[(E)-2-(5,6,7,8-tetrahydro-5,5,8,8-tetramethyl-2-naphthalenyl)-1-propenyl]benzoic acid;
4-(5-methoxymethyl-5-methyl-2,3,4,5-tetrahydro-1-benzo[b]oxepin-8-yl-ethynyl)-benzoic acid;
4-(5-ethoxymethyl-5-methyl-2,3,4,5-tetrahydrobenzo[b]oxepin-8-ylethynyl)-benzoic acid;
4-(5-methyl-5-propoxymethyl-2,3,4,5-tetrahydrobenzo[b]oxepin-8-ylethynyl)-benzoic acid;
(E)-4-[2-(5-methoxymethyl-5-propyl-2,3,4,5-tetrahydrobenzo[b]oxepin-8-yl)-vinyl]-benzoic acid;
(E)-4-[2-(5-methoxymethyl-5-methyl-2,3,4,5-tetrahydrobenzo[b]oxepin-8-yl)-vinyl]-benzoic acid;
(E)-4-[2-(5-methyl-5-propoxymethyl-2,3,4,5-tetrahydrobenzo[b]oxepin-8-yl)-vinyl]-benzoic acid;
4-(5-methoxymethyl-5-methyl-2,3,4,5-tetrahydrobenzo[b]thiepin-8-ylethynyl)-benzoic acid;
4-(5-ethoxymethyl-5-methyl-2,3,4,5-tetrahydrobenzo[b]thiepin-8-ylethynyl)-benzoic acid;
(E)-4-[2-(5-ethoxymethyl-5-methyl-2,3,4,5-tetrahydrobenzo[b]thiepin-8-yl)-vinyl]-benzoic acid;
(E)-4-[2-(5-methoxymethyl-5-propyl-2,3,4,5-tetrahydrobenzo[b]thiepin-8-yl)-vinyl]-benzoic acid;
4-(4-methoxymethyl-4-methyl-chroman-6-ylethynyl)-benzoic acid;
(E)-4-[2-(4-methoxymethyl-4-methyl-chroman-6-yl)-vinyl]-benzoic acid; or
any combination thereof.

Additional RARγ agonists are described in WO 2001030326; WO 2001014360; and Shimono et al. (Nat. Med. 17: 454-460 (2011)), which are specifically incorporated herein by reference in their entireties.

Agonists RARγ can be identified or evaluated by transactivation assays. The term “transactivation” refers to the ability of a retinoid to activate the transcription of a gene where the gene transcription is initiated by the binding of a ligand (e.g., agonist) to the RARγ. Determining the ability of a compound to transactivate a retinoic acid receptor can be performed by methods known to those of skill in the art. Examples of such methods are found in Bernard et al, Biochem. Biophys. Res. Commun., 186: 977-983 (1992) and C. Apfel et al, Proc. Nat. Sci. Acad. (USA), 89: 7129-7133 (1992).

In some embodiments, the RARγ agonist is a RARγ selective agonist, A RARγ selective agonist refers to a compound that is able to selectively bind to the RARγ receptor and promote RARγ activation. RARγ selective agonists will bind to the RARγ receptor at significantly lower concentrations (>10 fold selectivity, preferable 50 to 100 fold selectivity) than the RARα and RARβ receptors.

The RARγ agonist can, for example, be all-trans-retinoic acid

Characteristics of the Schwann Cells

The β-mercaptoethanol/retinoic acid protocol for Schwann cell differentiation developed by Dezawa et al (2001) for rat marrow stromal cells was also successful when used with rat adipose-derived stem cells (Kingham et al, 2007), but was not applicable to the human hEPI-NCSC. Retinoic acid treatment for 3 days (Dezawa et al, 2001) or 5 days (data not shown) was not conducive to highly efficient Schwann cell differentiation in the methods described herein, as judged by cell morphology and marker expression. Instead, prolonged retinoic acid treatment was used. The reason for this discrepancy between rodent and human cells is not obvious, as many signalling pathways that regulate cell differentiation are affected by retinoic acid.

The methods described herein induce SOX10 expression in the appropriate cellular location within hEPI-NCSC to achieve efficient differentiation. SOX10 is a key regulatory gene that is involved not only in neural crest formation, but also in Schwann cell fate decision and Schwann cell differentiation. SOX10 is also involved in regulating expression of the myelin proteins, p-zero (P0) and myelin basic protein (MBP). In contrast, SOX10 function must be shut-off for neuronal differentiation to proceed (Rehberg et al, 2002).

EPI-NCSC, reside in hair follicles and are the direct descendants of the embryonic neural crest. However, the gene expression profiles of embryonic and adult neural crest stem cells are not identical (Hu et al, 2006). SOX10 is expressed and functional in the embryonic neural crest. In hEPI-NCSC, SOX10 immunoreactivity was primarily localised in the cytoplasm. However, the majority of hEPI-NCSC cells also showed robust nuclear localisation after enhanced WNT and SHH signalling.

Similar to the in vivo situation, nuclear but not cytoplasmic SOX10 immunoreactivity decreased at later stages of in vitro differentiation of hEPI-NCSC. Enhanced WNT and SHH signalling may facilitate entry and exit to and from the nucleus by SOX10, which is a shuttle protein and such continuous shuttling is needed for SOX10 function (Rehberg et al, 2002). It is not known why SOX10 resides at least part time in the cytoplasm but is thought to involve post-translational modification, which over time gets lost during nuclear localisation. Thus nuclear entry/exit of SOX10 may represent a fast-acting regulatory system for SOX10 function (Rehberg et al, 2002).

Here, enhancing the WNT and SHH pathways resulted in a Schwann cell type that structurally and functionally resembles myelinating Schwann cells. In the absence of SHH and CHIR99021, Schwann cells expressed intense GFAP immunoreactivity, which is characteristic of non-myelinating Schwann cells.

SOX10 regulates the KROX20 gene, a key Schwann cell-specific transcription factor (Reiprich et al, 2010). Persistent KROX20 expression is characteristic of myelinating Schwann cells (Allodi et al, 2012). MBP and P-zero expression is also characteristic of myelinating Schwann cells. In contrast, p75NTR and GFAP are abundantly expressed in non-myelinating Schwann cells (Allodi et al, 2012).

As described herein, hEPI-NCSC derived Schwann cells expressed strong nuclear KROX20 immunoreactivity throughout the culture period. While progressing through differentiation, all cells also became intensely immunofluorescent for MBP and P-zero, and such gene expression was up-regulated. SOX10 and KROX20 synergistically activate MBP expression (Marathe et al., PLoS One 8(7):e69037 (2013)). No heterogeneity was observed in regard to P-zero expression in culture. In contrast, towards the end of the culture period only approximately 50% of cells were highly intensely MPB immunoreactive, whereas the remaining cells showed lower intensity of fluorescence. In co-culture with DRG neurons, however, MPB immunofluorescence was strong in all cells (data not shown), illustrating reciprocal influences between neurites and Schwann cells. In contrast, p75NTR immunoreactivity was very high at the onset of differentiation of hEPI-NCSC into Schwann cells, but intensity of immunofluorescence decreased with progressing differentiation in culture.

GFAP immunoreactivity was not detected initially in hEPI-NCSC subjected to the methods described herein and was present at modest levels only at later times of in vitro differentiation. However, in the absence of retinoic acid, SHH and CHIR99021, all cells were robustly GFAP immunoreactive, and SOX10 was localised in the cytoplasm.

Gene expression profiling confirmed that as in vitro differentiation progressed, Schwann cells reduced expression of transcripts characteristic for neural crest stem cells, including genes involved in cell migration, cell proliferation, and cell invasiveness. Instead, there was a concomitant increase in the expression of the myelin basic protein gene, genes involved in cholesterol metabolism, and in the expression of transcripts of relevant neurotrophins and other relevant growth factors. Importantly, gene expression profiling showed expression of transcripts for the JUN gene, a transcription factor in Schwann cells that is needed for nerve repair, as it regulates the molecular re-programming involved in turning mature Schwann cells into the regenerative Schwann cell type after nerve injury (Arthur-Farraj et al, 2012). Overall, the results described herein shows that hEPI-NCSC derived Schwann cells expressed a panel of markers characteristic for myelinating Schwann cells that are capable of supporting nerve regeneration.

Close alignment and interaction of the EPI-NCSC derived Schwann cells with neurites illustrates that the Schwann cells are functionally active. Schwann cells exhibited multiple protrusions that grew towards bundles of neurites and in some cases formed very close associations between the membranes of Schwann cells and neurites as observed at the ultrastructural level. Myelination of rodent DRGs by human Schwann cells in co-culture has not previously been reported in the literature and is thought not to occur.

Notably, hEPI-NCSC derived Schwann cells expressed transcripts for neurotrophic factors essential for nerve regeneration and expressed by Schwann cells in vivo during nerve regeneration. The genes that were expressed in the hEPI-NCSC derived Schwann cells included NGF (see, Rush, 1984, for information on NGF), NT-4/5 (Funakoshi et al, 1993), BDNF (Meyer et al, 1992), FGF2 (Ornitz and Ito 2001), FGF5 (McGeachie et al, 2001), LIF (Kurek et al. 1996) and IL-6 (Bolin et al, 1995).

One aspect for successful nerve regeneration includes cues from Schwann cells that promote angiogenesis in the injured microenvironment. VEGFa is a key angiogenic factor expressed in hEPI-NCSC, and VEGFa transcripts were up-regulated 6-fold in hEPI-NCSC derived Schwann cells. Hence, the hEPI-NCSC derived Schwann cells may contribute to angiogenesis in vivo. Others have shown that mouse EPI-NCSC (mEPI-NCSC; Hu et al, 2010) and dog/canine EPI-NCSC (cEPI-NCSC; McMahill et al, submitted) promote angiogenesis in vivo in models of spinal cord injury in mice and dogs.

The available data show that hEPI-NCSC are multipotent human adult stem cells that are readily accessible and obtained in the bulge of hair follicles. Due to their migratory ability, hEPI-NCSC can be isolated as a highly pure population of multipotent stem cells, and they can be expanded rapidly ex vivo. The results described herein illustrate that hEPI-NCSC can be differentiated into mammalian Schwann cells rapidly, with high efficiency and without the need for genetic manipulation or purification. The EPI-NCSC-derived Schwann cells expressed markers of myelinating Schwann cells that are conducive to nerve repair. Previous studies by the inventor and co-workers have shown that grafts of mouse EPI-NCSC in the mouse spinal cord (Hu et al, 2010), of canine EPI-NCSC in the canine spinal cord in a teratoma assay (McMahill et at. submitted) and of human hEPI-NCSC in the striatum of athymic rats (MSB, unpublished data) did not form tumours, which is a hallmark of adult stem cells. For these reasons combined, the EPI-NCSC derived human Schwann cells described herein are a valuable resource not only for translational research but also for treatment of a variety of neuronal diseases and disorders.

### Treatment

The EPI-NCSC-derived Schwann cells described herein can be used for regeneration of peripheral nerves or central nerves. Therefore, one aspect of the invention is the EPI-NCSC-derived Schwann cells themselves. Due to their different induced differentiation histories, they are artificially modified cells and are not products of nature.

One aspect of the invention includes a pharmaceutical composition that includes EPI-NCSC-derived Schwann cells and a pharmaceutically acceptable carrier. Such a composition is described in more detail below and can be used for treatment of a variety of neuronal diseases and disorders.

The EPI-NCSC-derived Schwann cells of the invention are suitable for autologous or allogenic transplantation. Cells of the nervous system are generally not as susceptible to immune system attack, but an immune response is possible when Schwann cells are administered in various loci (e.g., when stem cells differentiate they are no longer immune privileged). Rejection problems can therefore be avoided by using EPI-NCSC donor cells with matching histocompatibility antigens. The pharmaceutical composition may also contain common pharmaceutically acceptable carriers, physiologically acceptable buffers, salts, excipients and the like. The composition may be injected into the affected site directly or administered in a device, scaffold, hydrogel, or as a sustained release formulation. For example, the EPI-NCSC-derived Schwann cells can populate the interior surface of a hollow tube-like scaffold for transplantation at the site of a severed central or peripheral nerve. In another example, the cells can be incorporated into or onto a hydrogel scaffold for transplantation at the site of a severed central or peripheral nerve.

Although peripheral nerves intrinsically are capable of regeneration, it is known that they cannot regenerate over gaps of several centimeters. However, use of the EPI-NCSC-derived Schwann cells either as a composition or in a scaffold, hydrogel, device, or sustained release formulation can facilitate regeneration of nerves across such gaps.

Central nerve conditions wherein reconstruction is considered impossible encompass a wide gamut of different conditions, including injury-related spinal cord damage or cerebrovascular damage and diseases ranging from blinding glaucoma to degenerative diseases such as Parkinson's, which have a high estimated incidence rate among the population. The EPI-NCSC-derived Schwann cells may be used for regeneration of many and various types of central nerves. Research on methods of neural regeneration for the aforementioned conditions is an urgent social need, and the present invention is believed to have direct application for the human body.

The EPI-NCSC-derived Schwann cells can therefore be employed in a method of treating a subject with a neuronal disease or injury.

Examples of diseases and injuries that can be treated using the EPI-NCSC-derived Schwann cells and compositions (containing any of the compounds described herein with or without EPI-NCSC-derived Schwann cells include Guillain-Barre syndrome, multiple sclerosis, Amyotrophic lateral sclerosis (ALS), chronic inflammatory demyelinating polyradiculoneuropathy (CIDP). Experimental allergic encephalomyelitis (EAE), Alzheimer's disease, Parkinson's disease, primary lateral sclerosis, Progressive bulbar palsy, Pseudobulbar palsy, Primary lateral sclerosis (PLS), Progressive muscular atrophy, Spinal muscular atrophy (SMA), including Type I (also called Werdnig-Hoffmann disease), Type II, Type III (Kugelberg-Welander disease), Fazio-Londe disease, Huntington's disease, Kennedy's disease also known as progressive spinobulbar muscular atrophy, hereditary spastic paraplegia (HSP), congenital SMA with arthrogryposis, Post-polio syndrome (PPS), traumatic spinal cord injury, progressive pseudobulbar palsy, progressive muscular atrophy, progressive bulbar palsy, postpolio syndrome, stroke, head trauma, spinal cord injury, and the like.

Diseases and conditions that can be treated include those that occur as a consequence of genetic defect, physical injury, environmental insult or conditioning, bad health, obesity and other disease risk factors commonly known by a person of ordinary skill in the art.

Efficacy of treatment can be monitored by clinically accepted criteria and tests, which include for example, using Electromyography (EMG), which is used to diagnose muscle and nerve dysfunction and spinal cord disease, and measure the speed at which impulses travel along a particular nerve. EMG records the electrical activity from the brain and/or spinal cord to a peripheral nerve root (found in the arms and legs) that controls muscles during contraction and at rest. One can also monitor efficacy of treatment using a nerve conduction velocity study to measure electrical energy to test the nerve's ability to send a signal, as well as laboratory screening tests of blood, urine, as well as magnetic resonance imaging (MRl), which uses computer-generated radio waves and a powerful magnetic field to produce detailed images of body structures including tissues, organs, bones, and nerves to detect and monitor degenerative disorders. In some embodiments, efficacy of treatment can also be assessed by a muscle or nerve biopsy can help confirm nerve disease and nerve regeneration. A small sample of the muscle or nerve is removed under local anesthetic and studied under a microscope. The sample may be removed either surgically, through a slit made in the skin, or by needle biopsy, in which a thin hollow needle is inserted through the skin and into the muscle. A small piece of muscle remains in the hollow needle when it is removed from the body. In some embodiments, efficacy of treatment can also be monitored by a transcranial magnetic stimulation to study areas of the brain related to motor activity.

### Administration of EN-NCSG-Derived Schwann Cells

The EPI-NCSC-derived Schwann cells generated as described herein can be employed for tissue reconstitution or regeneration in a human patient, a mammalian subject, or other subjects in need of such treatment. Human patients are also mammalian subjects. The cells are administered in a manner that permits them to graft or migrate to a diseased or injured tissue site and to reconstitute or regenerate the functionally deficient area. Devices are available that can be adapted for administering cells, for example, into the spinal cord or other parts of the central or peripheral nervous system.

EPI-NCSC-derived Schwann cells can be administered to reconstitute the neuronal cell population in the spinal cord, brain, or at an alternative desired location. The cells may be administered to a recipient by local injection, or by systemic injection. In some embodiments, the cells can be administered parenterally by injection into a convenient cavity or by intramuscular injection.

Many cell types are capable of migrating to an appropriate site for regeneration and differentiation within a subject. To determine the suitability of cell compositions for therapeutic administration, the cells can first be tested in a suitable animal model.

At one level, cells are assessed for their ability to survive and maintain their phenotype in vivo. Cells can also be assessed to ascertain whether they migrate to diseased or injured sites in vivo, or to determine an appropriate number of dosage of cells to be administered. Cell compositions can be administered to immunodeficient animals (such as nude mice, or animals rendered immunodeficient chemically or by irradiation). Tissues can be harvested after a period of regrowth, and assessed as to whether the administered cells or progeny thereof are still present, are alive, and/or have migrated to desired or undesired locations.

Injected cells can be traced by a variety of methods. For example, cells containing or expressing a detectable label (such as green fluorescent protein, or beta-galactosidase) can readily be detected. The cells can be pre-labeled, for example, with BrdU or [³H] thymidine, or by introduction of an expression cassette that can express green fluorescent protein, or beta-galactosidase. Alternatively, the EPI-NCSC-derived Schwann cells can be labeled with magnetisable beads and traced live by MRI (McMahill et al., Stem Cells Transl Med. 4(10):1173-86 (October 2015)), or they can be detected by their expression of a cell marker that is not expressed by the animal employed for testing (for example, a human-specific antigen). The presence and phenotype of the administered population of EPI-NCSC-derived Schwann cells can be assessed by fluorescence microscopy (e.g., for green fluorescent protein, or beta-galactosidase), by immunohistochemistry (e.g., using an antibody against a human antigen), by ELISA (using an antibody against a human antigen), or by RT-PCR analysis using primers and hybridization conditions that cause amplification to be specific for selected nucleic acid segments.

A number of animal models for testing in models of motor neuron diseases are available for such testing, for example as the SOD1(G93A) mutant mouse and SMA (B6.129-Smnl^{tmlJmeJ}) mouse models from Jackson laboratories.

An EPI-NCSC-derived Schwann cell population can be introduced by injection, catheter, implantable device, or the like. A population of EPI-NCSC-derived Schwann cells can be administered in any physiologically acceptable excipient or carrier that does not adversely affect the cells.

A population of EPI-NCSC-derived Schwann cells can be supplied in the form of a pharmaceutical composition. Such a composition can include an isotonic excipient prepared under sufficiently sterile conditions for human or mammalian administration. For general principles in medicinal formulation, the reader is referred to Cell Therapy: Stern Cell Transplantation, Gene Therapy, and Cellular immunotherapy, by G. Morstyn & W. Sheridan eds, Cambridge University Press, 1996; and Hematopoietic Stern Cell Therapy, E. D. Ball, J. Lister & P. Law, Churchill Livingstone, 2000. The choice of the cellular excipient and any accompanying constituents of the composition that includes a population of EPI-NCSC-derived Schwann cells can be adapted to optimize administration by the route and/or device employed.

A composition that includes a population of EPI-NCSC-derived Schwann cells can also include or be accompanied by one or more other ingredients that facilitate engraftment or functional mobilization of the EPI-NCSC-derived Schwann cells. Suitable ingredients include matrix proteins that support or promote adhesion of the EPI-NCSC-derived Schwann cells, or complementary cell types, such as glial and/or muscle cells. In another embodiment, the composition may include physiologically acceptable matrix scaffolds or hydrogels. Such physiologically acceptable matrix scaffolds and hydrogels can be resorbable and/or biodegradable.

The population of EPI-NCSC-derived. Schwann cells generated by the methods described herein is typically very pure. For example, virtually 100% of the cells produced by the methods described herein stain for immunological markers of Schwann cells. In various embodiments, the cell populations produced herein are at least 80% Schwann cells, or at least 85% Schwann cells, or at least 90% Schwann cells, or at least 92% Schwann cells, or at least 93% Schwann cells, or at least 95% Schwann cells, or at least 96% Schwann cells, or at least 97% Schwann cells, or at least 98% Schwann cells, or at least 99% Schwann cells, or at least 99.5% Schwann cells.

However, the population of EPI-NCSC-derived Schwann cells generated by the methods described herein can include low percentages of non-neuronal cells (e.g., EPI-NCSC). For example, a population of EPI-NCSC-derived Schwann cells to added to a composition for administration to subjects can have less than about 90% non-Schwann cells, less than about 85% non-Schwann cells, less than about 80% non-Schwann cells, less than about 75% non-Schwann cells, less than about 70% non-Schwann cells, less than about 65% non-Schwann cells, less than about 60% non-Schwann cells, less than about 55% non- Schwann cells, less than about 50% non-Schwann cells, less than about 45% non-Schwann cells, less than about 40% non-Schwann cells, less than about 35% non-Schwann cells, less than about 30% non-Schwann cells, less than about 25% non-Schwann cells, less than about 20% non-Schwann cells, less than about 15% non-Schwann cells, less than about 12% non-Schwann cells, less than about 10% non-Schwann cells, less than about 8% non-Schwann cells, less than about 6% non-Schwann cells, less than about 5% non-Schwann cells, less than about 4% non-Schwann cells, less than about 3% non-Schwann cells, less than about 2% non-Schwann cells, or less than about 1% non-Schwann cells.

Supplementary factors can be included in the compositions and/or in a cell culture media containing any of the compositions, compounds or agents described herein. Examples of such supplementary factors include bone morphogenic protein (BMP)-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-I5, brain derived neurotrophic factor, ciliary neutrophic factor, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor (FGF) 4, fibroblast growth factor 5, fibroblast growth factor 6, _fibroblast growth factor 7, fibroblast growth factor 8, fibroblast growth factor 8b, fibroblast growth factor 8c, fibroblast growth factor 9, fibroblast growth factor 10, fibroblast growth factor (acidic), fibroblast growth factor (basic), growth related protein, growth related protein α, growth related protein β, growth related protein γ, heparin binding epidermal growth factor, hepatocyte growth factor, insulin-like growth factor I, insulin-like growth factor II, insulin-like growth factor binding protein, keratinocyte growth factor, leukemia inhibitory factor, neurotrophin-3, neurotrophin-4, placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, pre-B cell growth stimulating factor, stem cell factor, transforming growth factor a, transforming growth factor β, transforming growth factor β1, transforming growth factor 01.2, transforming growth factor 132, transforming growth factor β3, latent transforming growth factor β transforming growth factor β binding protein I, transforming growth factor 13 binding protein II, transforming growth factor β binding protein III, and vascular endothelial growth factor.

Exemplary cytokines can be included such as interleukin (IL)-1, IL-2, IL-3, IL-4, IL-5, IL-6, IL-7, IL-8, IL-9, IL-10, IL-11, IL-12, IL-13, 1L-14, IL-15, IL-16, 1L-17, IL-18, interferon (IFN), IFN-γ, tumor necrosis factor (TNF), TNF1, TNF2, TNF-α, macrophage colony stimulating factor (M-CSF), granulocyte-monocyte colony stimulating factor (GM-CSF), granulocyte colony stimulating factor (G-CSF), megakaryocyte colony stimulating factor (Meg-CSF)-tbronibopoietin, stem cell factor, and erythropoietin. Chemokines can also be included such as IP-10 and Stromal Cell-Derived Factor 1α.

Exemplary hormones contemplated for inclusion in the compositions and/or cell culture media described herein can include, but are not limited to, steroid hormones and peptide hormones, such as insulin, somatostatin, growth hormone, hydrocortisone, dexamethasone, 3,3′,5-Triiodo-L-thyronine, and L-Thyroxine.

### Kits

A variety of kits are described herein that include any of the compositions, compounds and/or agents described herein. The compounds and/or agents described herein can be packaged separately into discrete vials, bottles or other containers. Alternatively, any of the compounds and/or agents described herein can be packaged together as a single composition, or as two or more compositions that can be used together or separately. The compounds and/or agents described herein can be packag in appropriate ratios and/or amounts to facilitate conversion of selected cells across differentiation boundaries to form neuronal cells.

A kit is described herein for culture of cells in vitro that can include any of the compositions, compounds and/or agents described herein, as well as instructions for using those compositions, compounds and/or agents, Some kits can include a cell culture or cell media that includes any of the compositions, compounds and/or agents described herein.

The kits can include one or more sterile cell collection devices such as a swab, skin scrapping device, a needle, a syringe, and/or a scalpel. The kits can also include antibodies for detection of various cell markers, including EPI-NCSC-derived Schwann cell markers. Such antibodies can include anti-myelin protein zero antibodies, anti-myelin basic protein (MBP) antibodies, anti-S100 antibodies, anti-glial fibrillary acidic protein (GFAP) antibodies, anti-SOX10 antibodies, anti-p75NTR antibodies, anti-human nuclei antibodies, anti-neurofilament antibodies, anti-early growth response 2 (KROX20; ERG2) antibodies, and combinations thereof. Secondary antibodies for detection of any of the foregoing cell marker antibodies can also be included in the kits. The antibodies can be labeled so that a detectable signal can be observed when the antibodies form a complex with the cell markers, including EPI-NCSC-derived Schwann cell marker(s).

The instructions can include guidance for culturing EPI-NCSC for a time and under conditions sufficient to differentiate those cells into EPI-NCSC-derived Schwann cells. For example, the instructions can describe amounts of the compositions, compounds and/or agents described herein to add to cell culture media, times sufficient to convert cells to the neuronal lineage, maintenance of appropriate cell densities for optimal conversion, and the like. For example, the instructions can describe procedures for rehydration or dilution of the compositions, compounds and/or agents described herein. When a kit provides a cell culture medium containing sonic of the compositions, compounds and/or agents described herein, the instructions can describe how to add other compounds and/agents. The instructions can also describe how to convert the selected cells to neuronal progenitor cells or to mature neuronal cells.

The instructions can also describe procedures for detecting EPI-NCSC-derived Schwann cell markers by use of the antibodies against those markers so that the extent of conversion and/or differentiation can be assessed.

Another kit is also described herein that includes any of the compositions, compounds and/or agents described herein for therapeutic treatment of a subject. The kit can include any of the compositions, compounds and/or agents described herein, as well as instructions for administering those compositions, compounds and/or agents. Such instructions can provide the information described throughout this application The kit can also include cells. For example, the kit can include EPI-NCSC-derived Schwann cells that have been treated by the methods described herein and that are ready for administration.

The cells, compositions and/or compounds can be provided within any of the kits in a delivery device. Alternatively a delivery device can be separately included in the kit(s), and the instructions can describe how to assemble the delivery device prior to administration to a subject. The delivery device can provide a scaffold or hydrogel for cell growth and/or a matrix for controlled release of any of the compositions, compounds or agents described herein.

Any of the kits can also include syringes, catheters, scalpels, sterile containers for sample or cell collection, diluents, pharmaceutically acceptable carriers, and the like.

The kits can provide other factors such as any of the supplementary factors described herein for the compositions in the preceding section.

### Definitions

An isolated EPI-NCSC population can be substantially pure. The term “substantially pure”, as used herein means that at the onset of emigration from the hair follicle, the isolated cell population of neural crest cells includes greater than 70%, or at least 83%, or at least 90% multipotent neural crest stem cells. In other words, the term “substantially pure” refers to a population of progenitor cells that contain fewer than about 30%, or fewer than about 17%, or fewer than about 10% of lineage committed cells in the original expansion and isolated population prior to subsequent culturing and expansion. This high level of purity can be obtained by following the methods set forth herein or those described in U.S. Pat. No. 8,030,072 without any need for additional purification steps to further purify the cells.

As used herein the term “EPI-NCSC” refers to those cells that originate from the neural folds of the neuroepithelium and reside in the epidermal bulge region of non-embryonic hair follicle. Soon after emigration from the embryonic neural tube EPI-NCSC invade the somatic ectoderm and become located in the bulge of hair follicles. hEPI-NCSC is human EPI-NCSC.

As used herein the term “non-embryonic” stem cells refer to stem cells from adults, juveniles or new-boors. No one was harmed in the process. No embryos or non-born fetuses are involved in the collection of the cells or their transplantation. Further, the stem cells are an undifferentiated cell found in a differentiated tissue (e.g., epidermis) that can renew itself and (with certain limitations) differentiate to yield all the specialized cell types neural crest derivatives, as well as non-crest cells.

As used herein, the “neural crest” is a transient embryonic tissue (a band of cells that extend lengthwise along the neural tube of an embryo) that contains highly plastic multipotent stem cells, which are capable of undergoing self-renewal and give rise to cells that form the cranial ganglia, spinal ganglia, the autonomic and enteric nervous systems, as well as becoming odontoblasts, which form the calcified part of the teeth, as well as many other cell types and tissues.

As used herein the term “neural crest derivatives” include the autonomic nervous system (sympathetic and parasympathetic), the enteric nervous system, most primary sensory neurons, smooth muscle cells of the cardiac outflow tract and great vessels of the head and neck, endocrine cells (adrenal medulla, calcitonin-proclucing-cells of the thyroid), pigment cells of the skin and internal organs, and the following cranio-facial structures: connective tissue, cartilage and bone of the face, forehead and ventral neck, tooth papillae, meninges, striated muscle of the eve and the stromal cells of the cornea. Under the culture conditions described herein EPI-NCSC can differentiate into all major neural crest derivatives, including neurons, Schwann cells, cartilage cells, bone cells, myofibroblasts and pigment cells. This observation indicates the EPI-NCSC can express all major neural crest derivatives.

A “somatic cell” is a cell forming the body of an organism. Somatic cells include cells making up organs, skin, blood, bones and connective tissue in an organism, but not germ cells.

Cells can be from, e.g., human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human animal.

As used herein, the term “totipotent” means the ability of a cell to form all cell lineages of an organism. For example, in mammals, only the zygote and the first cleavage stage blastomeres are totipotent.

As used herein, the term “pluripotent” means the ability of a cell to form all lineages orf the body or soma (i.e., the embryo proper). For example, embryonic stem cells are a type of pluripotent stem cells that are able to form cells from each of the three germs layers, the ectoderm, the mesoderm, and the endoderm.

As used herein the term “multipotent” refers to stem cells that have the ability to develop into more than one cell type of the body.

As used herein, the terms “subject” or “patient” refers to any animal, such as a domesticated animal, a zoo animal, or a human. The “subject” or “patient” can be a mammal or bird, such as a domesticated, zoo, or endangered wild animal. Examples include dogs, cats, horses, livestock, and humans. Additional specific examples of “subjects” and “patients” include, but are not limited to, individuals with a neuronal disease or disorder, and individuals with neuronal disorder-related characteristics or symptoms.

As used herein the term “plastic” or “plasticity” refers to the ability of stem cells from one adult tissue to generate two or more differentiated types of progeny. Also, the phrase “high degree of physiological plasticity” refers to the fact that neural crest cells are capable of generating a wider range of cell types that are typical for ectodermal derivatives (e.g., neurons, Schwann cells, corneal stroma, meninges, pigment cells, endocrine cells), and mesodermal derivatives (e.g., cartilage, bone, tooth papillae, myofibroblast, connective tissue) in relative to other stem cells of non-embryonic origin.

As used herein the term “explant” refers to a portion of an organ epidermis) taken from the body and grown in a defined medium.

As used herein the phrase “adherent explants” refers to bulge regions that have been explanted to coated culture plates (e.g., CELLstart™-coated plates). Under these conditions the bulge regions adhere to the collagen substratum within one hour, at which time the culture medium described herein is added, and the emigrating cells are highly migratory.

As used herein the phrase “adherent cells” refers to cells that are cultured while adhering to a substratum in either tissue culture plastic plates, flasks, or on microcarriers. The substratum can include a culture substrate or be coated with a substance such as collagen.

As used herein the phrase “highly proliferative in in vitro culture” refers to the fact that the cells have a population doubling time that initially in primary explants is approximately six hours, and later in expansion culture of approximately 24-30 hours.

As used herein the phrase “highly motile” refers to the fact that the cells can emigrate from the bulge explants and translocate randomly in a culture plate for clonal cultures, whereas epidermal stem cells remain near the explant.

As used herein the phrase “at the onset of emigration from the hair bulge explant follicle” refers to the point in time at which the first EPI-NCSC emerge from the bulge explants, usually 2-4 days post-explantation explantation for mouse EPI-NCSC and 6-10 days for human EPI-NCSC.

As used herein, the “bulge region” is a defined, multi-layered area of the epidermis of a hair follicle, or in an animal whisker follicle. It also exists in hair follicles of other parts of a mammal. The bulge region is more elongated in human hair follicles than in most animal follicles.

The term “culture substrates” as used herein refers to any surface that can be used for effectively isolating and culturing the EPI-NCSC in adherent culture. These can include for example, collagen-coated, gelatin-coated, poly-lysine-coated, or CELLstart™-coated substrates. The vessels that can have culture substrates include tissue culture Petri dishes, culture flasks, glass coverslips, glass-bottom plates, and Nunc Cell Factory vessels, which are all effective at culturing small quantities of attachment dependent cells. Culture substrates also include microcarriers, fiber beds, hollow fiber cartridges, hydrogels, and stacked plate modules used for attachment dependent cell culture scale up. For example, microcarriers are tiny beads or particles with a surface chemistry that facilitates attachment and growth of anchorage-dependent cells in cell culture processes for scale-up purposes. There are numerous types of microcarriers, widely varying in their composition, size, shape and density including but not limited to HyQ® Spheres™ Microcarriers, PuraMatrix™ Peptide Hydrogels, collagen hydrogels, and others.

The term “about” can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The Examples describe some of the experimental work involved in the development of the invention and some of the characteristics of the EPI-NCSC-derived Schwann cells.

### EXAMPLE 1

### Materials and Methods

This Example describes some of the materials and methods used in developing the invention.

hEPI-NCSC Cultures

hEPI-NCSC were isolated from human hair follicles as described previously (Clewes et al 2011). Briefly, anagen hair follicles were dissected and the bulge section placed into adherent culture. hEPI-NCSC emigrating from bulge explants were subcultured, expanded for six days as described previously by the inventor and co-workers (Clewes et al 2011), and then cryopreseryed.

No Cd34 transcripts were observed in EPI-NCSC so isolated as detected by by RT-PCR. The lack of Cd34 expression sets EPI-NCSC apart from the nestin⁺/CD34⁺follicular cells described by Amoh et al. (Proc. Natl. Acad Sci U S A 102: 17734-38 (2005), as well as from hematopoietic stem cells, and from adipocyte stem cells.

Differentiation of hEPI-NCSC into Schwann Cells

hEPI-NCSC were seeded at 2,500-5,000 cells per CELLstart™-coated 35-mm culture plate and cultured at 36.8° C., 5% CO₂ and 5% O₂. At differentiation Day minus 3 (D-3), cells were placed in XP medium, which is NeuroCult XF medium (Stem Cell Technologies, Grenoble, France Cat# 05761) supplemented with 10 ng/ml rhFGF2 (R&D Systems, Abingdon, UK Cat# 233-FB), 20 ng/ml rhEGF (R&D Systems Cat# 236-EG), 1× ITS+3 (Sigma, Poole, UK Cat# 1-2771), 1% (v/v) FBS (HyClone, Thermo Fisher, Cramlington, UK Cat# SH30070.02), 1× GlutaMAX (Invitrogen, Cat# 35050-038), 1× Penicillin/Streptomycin (Sigma Cat# P0781) and 2.5 μg/ml Amphotericin B (Sigma Cat# A2942).

The cells in XP medium were treated with 500 ng/ml recombinant human sonic hedgehog (C2411) N-terminus (rhSHH) and 500 nM CHIR99021 for two days. At differentiation Day −1, the cells were subcultured. Cells were detached with TrypLE Select, centrifuged, and plated in XP medium at a density of 30,000 cells per 35 mm culture plate and 1,000 cells per poly-D-lysine and laminin-coated 13-mm diameter glass coverslip. The next day (differentiation Day 0), cells were cultured in Alpha-modified MEM in the presence of 10% FBS and 1 mM β-mercaptoethanol (βME) for 24 hours, which was followed by treatment with 35 ng/ml all-trans-retinoic acid according to the method of Dezawa et al (2001). At differentiation Day 3, the FBS concentration was reduced to 1%. Starting at differentiation Day 0, 10 μM SB431542 was added to the culture medium. Retinoic acid and SB431542 remained in the culture medium throughout the culture period. At Day 3, 10 ng/ml rhFGF2, 5 ng/ml recombinant human PDGF-BB (rhPDGF-BB), 5 μM forskolin, 200 ng/ml recombinant human neuregulin-1 (rhNRG) were added to the culture medium. The culture medium was exchanged every 48 hours.

### Dorsal Root Ganglion (DRC) Explants Culture

Mice ([C57BL/6-TgN(ACTbEGFP)]Osb]; ‘green mice’) (Okabe et al, 1997) and rats were handled in accordance to protocols approved by the Animal Care Committee of Newcastle University (project licence 60/3876). Whole mount DRG explants from E15.5 Wistar rat embryos were dissected as described by Päiväläinen et al. (2008) and Liu et al. (2011). Dissociated DRG cultures from adult ‘green’ mice were prepared according to Malin et al (2007) and Johnson et al (2001). To remove non-neuronal cells, DRG explants were treated with camptothecin (20 μM) three times for 48 hours each, on Days 2-5, 6-8, and 13-15.

Co-Cultures of hEPI-NCSC Derived Schwann Cells and DRG Neurons

At Day 28 of differentiation, hEPI-NCSC derived Schwann cells were detached with TrypLE and seeded onto camptothecin-treated DRG explants (at Day 17 of DRG explant culture) or dissociated DRG neuron cultures (at Day 10 of DRG explant culture) at 12,000 Schwann cells per 13-mm coverslip containing one DRG explant, and incubated for 21 days in co-culture medium. Co-culture medium consisted of alpha-modified MEM, penicillin/streptomycin, B27 supplement without retinoic acid, 5 ng/ml rhPDGF-BB, 5 μM forskolin, 10 ng/ml rhNRG, 50 μg/ml ascorbic acid, 20 ng/ml rhPDGF, 20 ng/ml GDNF, 20 ng/ml rhβNGF, 20 ng/ml rhNT3 and 10 μM SB431542.

### Indirect Innnunocytochemistry

Cultures were fixed with 4% paraformaldehyde, rinsed, blocked, and incubated overnight with primary antibodies in 0.1% Triton X100; rabbit anti-myelin protein zero antibody (1:100), rabbit anti-myelin basic protein (MBP) antibody (1:400), rabbit anti-S100 antibody (1:200), rabbit anti-glial fibrillar acidic protein (GFAP) antibody (1:200), goat anti-SOX10 antibody (1:100), rabbit anti-p75NTR antibody (1:500), mouse anti-human nuclei antibody (1:200), chicken anti-neurofilament H antibody (1:10,000), or mouse anti-early growth response 2 (KROX20; ERG2) antibody (1:100). The next day, cultures were rinsed exhaustively, incubated in secondary antibody for 2 hours, rinsed again and incubated with secondary antibodies (1:200 in PBS); Alexafluor-488 conjugated goat anti-rabbit IgG, Alexafluor-594 conjugated donkey anti-goat IgG, Alexafluor-488 conjugated donkey anti-rabbit IgG, Alexafluor-594 conjugated goat anti-rabbit IgG, Alexafluor-488 conjugated goat anti-chicken IgY, or Cy5 conjugated goat anti-mouse IgG. Finally, cultures on coverslips were mounted with Vectashield hard set mounting medium with 4′,6-diamidino-2-phenylindole (DAPI) and fluorescence observed with an Imager.Z1 fluorescence microscope or with a Nikon AIR Confocal Microscope. The list of reagents and sources is provided in supplemental Table 1.

### Electron Microscopy

Co-cultures of hEPI-NCSC derived Schwann cells with rat DRG explants were fixed for electron microscopy with 2.5% glutaraldehyde in 0.1 M Sorenson's phosphate buffer for one hour at room temperature. Subsequently, they were rinsed with Sorenson's phosphate buffer. The DRGs in centre of the co-culture was removed. The axons and Schwann cells remained attached to the culture well. Cell-covered well bottoms were isolated, shipped in buffer at room temperature and processed for electron microscopy. The cells on the well bases were post-fixed in 1% osmium tetroxide in PBS, dehydrated in a graded series of ethanol, embedded in a thin layer of Epon resin and polymerised for 48 h at 60° C. The region of interest was identified in a light microscope, cut out with a single edge razor blade, flat-embedded in Epon resin and polymerised as before. Ultrathin sections of 70 nm were cut with a Diatome diamond knife, mounted on pioloform/carbon films on copper slot grids and counter-stained with 4% uranyl acetate and Reynolds' lead citrate. Images were taken using a JEM 1400 transmission electron microscope equipped with an AMT XR60 digital camera.

RNA Isolation, Gene Expression Profiling and Real-Time Polymerase Chain Reaction (gPCR)

Total RNA was extracted from cells at differentiation Day −3 and Day 21 from snap-frozen homogenized cells using an AllPrep DNA/RNA/miRNA Universal Kit on a QIAcube platform (Qiagen), and eluted in 30 μl RNase free water. RNA purity was determined using an Agilent 2100 Bioanalyser (RIN values: Day −3 sample 9.9; Day 21 sample 10.0). Gene expression profiling was performed by the Genome Centre, University of London, on a Human HT-12 v4 array using the Illumina platform. The data are deposited in the NCBI Gene Expression Omnibus (see website at ncbi.nlm.nih.gov/geo/) under GEO accession number GSE61273. For qPCR, 1 μg total RNA was incubated with DNaseI. SuperScript III reverse transcriptase, Oligo(dT)₂₀ primer and dNTP mix were used to synthesis cDNA from the total RNA according to manufacturer's instructions. qPCR was performed using Fast SYBR Green Master mix exactly as we have described previously (Clewes et al., 2011). The qPCR conditions were as following: first step (95° C., 20 sec) and 40 cycles of denaturation (95° C., 15 sec) with final extension (60° C., 1 min) on a Taqman 7500 thermocycler. Ct values for targets were normalized to the average Ct value of GAPDH. Fold changes in expression were calculated using the ΔΔCt method.

### EXAMPLE 2

### Method for Differentiation of hEPI-NCSC Cells

Pilot experiments suggested that SOX10 preferentially confined to the cytoplasm in hEPI-NCSC was functionally inadequate for differentiation of hEPI-NCSC into Schwann cells. This Example describes a method for differentiating hEPI-NCSC cells

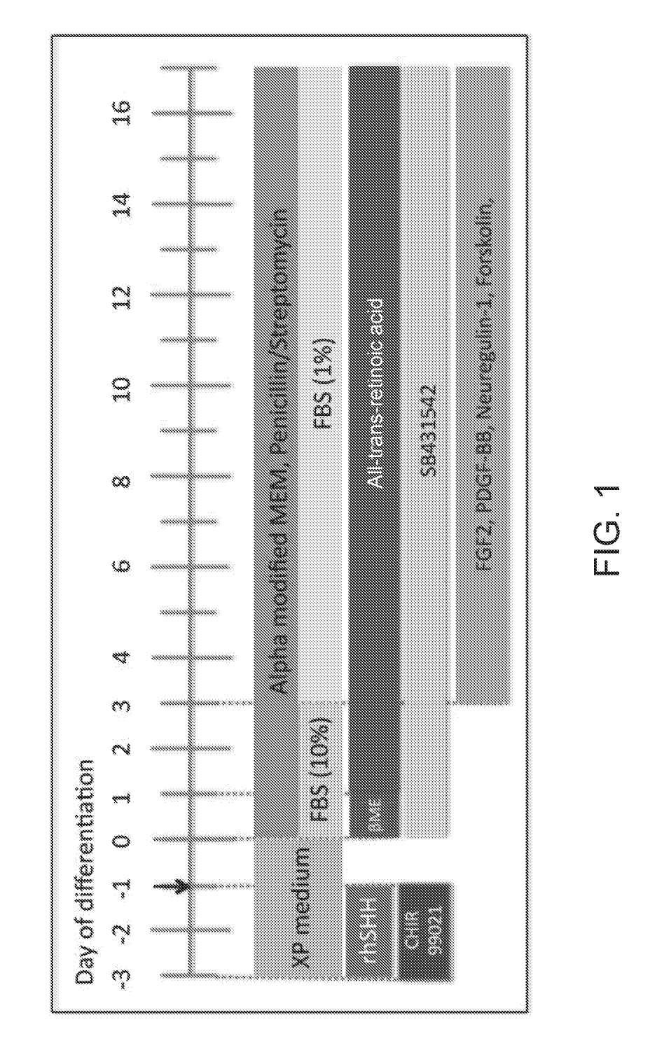

After ex vivo expansion, hEPI-NCSC cells were treated transiently with SHH and the a WNT signaling activator, CHIR99021, which lead to SOX10 localization in the nucleus and to highly efficient differentiation of hEPI-NCSC into Schwann cells (FIG. 1). At Day 0 of differentiation, cells were treated for 24 hours with β-mercaptoethanol, and all-trans-retinoic acid was added on differentiation Day 1 according to the method of Dezawa et al (2001). Neither the three-day treatment with retinoic acid, nor five days of exposure to retinoic acid were sufficient for differentiating hEPI-NCSC into Schwann cells as judged by cell morphology and marker expression. Retinoic acid was therefore left in the differentiation medium throughout the culture period without any detectable deleterious effects to the cells. At Day 0, SB431542, an inhibitor of transforming growth factor-beta 1 (TGFβ1) activin receptor-like kinases (ALKs), was also added to the culture medium (FIG. 1). Inhibition of TGFβ signaling with SB431542 throughout the differentiation period facilitated differentiation. On Day 3, FGF2, PDGF-BB, forskolin, and neuregulin-1 were added.

### EXAMPLE 3

### Morphology of Cells During Differentiation

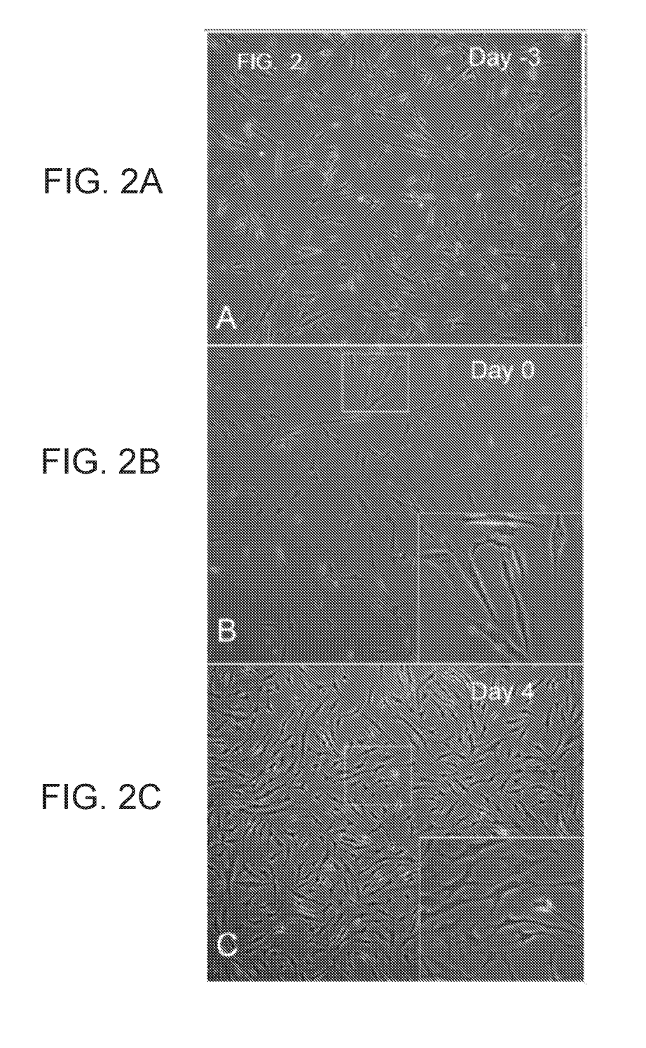

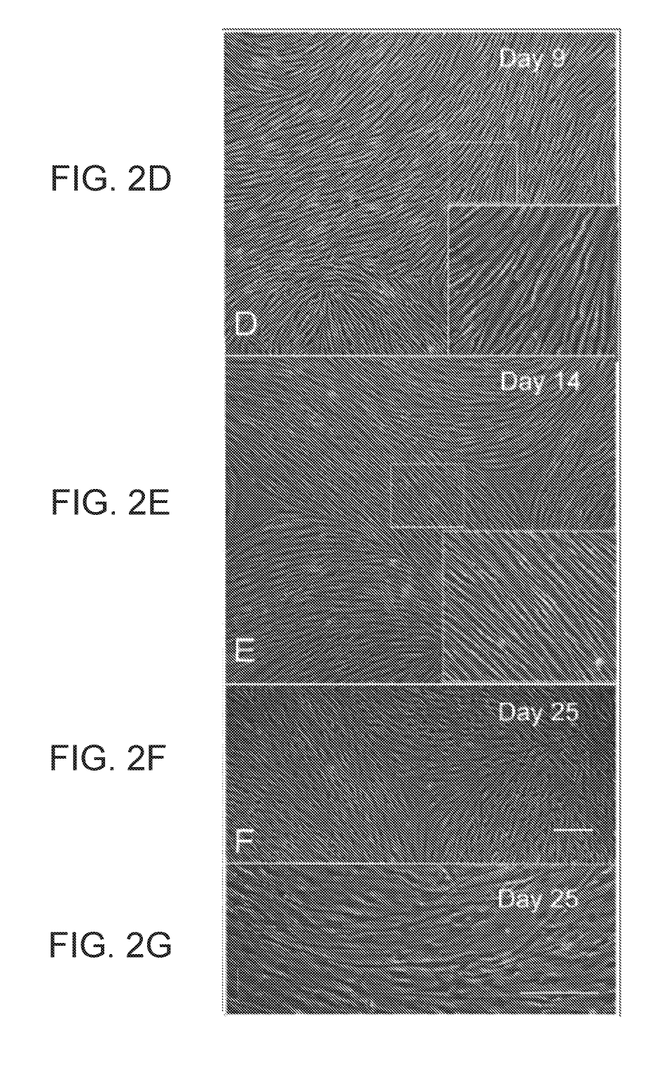

Prior to differentiation, hEPI-NCSC had the typical stellate morphology of neural crest stem cells (FIG. 2A), which remained unchanged after pre-treatment with SHH and CHIR99021 and sub-culture (FIG. 2B). By Day 4 of differentiation, cells became more elongated (FIG. 2C). By Day 9 cells had assumed the slender morphology of Schwann cells and they started to form swirls in the culture plate (FIG. 2D). The cells maintained this morphology for as long as they were kept in culture (up to 30 days; FIG. 2E, 2F, 2G). Under these conditions, cells continued to proliferate in differentiation culture until approximately days 9-14 of differentiation as judged by the presence of mitotic cells and increase in cell density. Schwann cells can be cryopreserved and re-cultured (data not shown).

### EXAMPLE 4

### Expression of Schwann Cell Markers

Cells were probed with antibodies against epitopes specific or characteristic for Schwann cells during treatment as described herein. Robust Schwann cell marker expression was detected. All cells were immunopositive for the neural crest stem cell and Schwann cell marker, SOX10, in the cytoplasm throughout the culture period (Table 1).

TABLE 1
Marker expression by immunocytochemistry

| | Epitope expression | | | | | | |
|---|---|---|---|---|---|---|---|
| Culture | (Percent of Total ± SEM) | | | | | | |
| Day | SOX10 | KROX20 | P75NTR | S100 | GFAP | MBP | P-zero |
| 0 | 100 | 100 | 100 | 100 | 0 | 100 | 100 |
| | (52.5 ± 8.1)* | (38.3 ± 10.1)* | | | | | |
| 4 | 100 | 100 | 100 | 100 | 0 | 100 | 100 |
| | (95.4 ± 1.4)*^{§} | (74.7 ± 5.6)*^{§} | | | | | |
| 9 | 100 | 100 | 100 | 100 | 0 | 100 | 100 |
| | (96.1 ± 0.8)*^{§} | (91.9 ± 0.8)*^{§} | | | | | |
| 14 | 100 | 100 | 100 | 100 | 0 | 100 | 100 |
| | (89.0 ± 2.5)*^{§} | (95.6 ± 1.2)*^{§} | | | | | |
| 19 | 100 | 100 | 100 | 100 | 100** | 100 | 100 |
| | (45.8 ± 3.7)* | (90.4 ± 2.1)*^{§} | | | | | |
| 30 | 100 | 100 | 100 | 100 | 100** | 100 | 100 |
| | (23.6 ± 2.4)*^{§} | (90.2 ± 2.1)*^{§} | | | | | |

*Percent of cells that also had nuclear marker expression in addition to cytoplasmic localisation.
^{§}Statistically significant difference from Day 0 value (p < 0.05; Dunnett's test)
**Intensity of fluorescence at level of detection only.

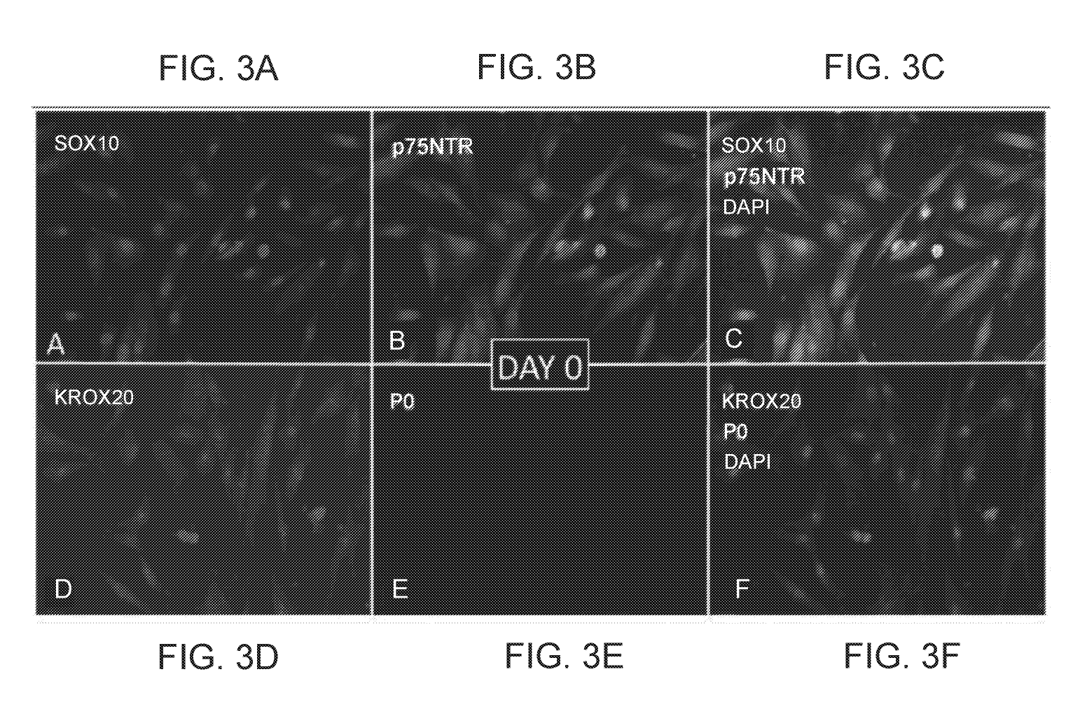

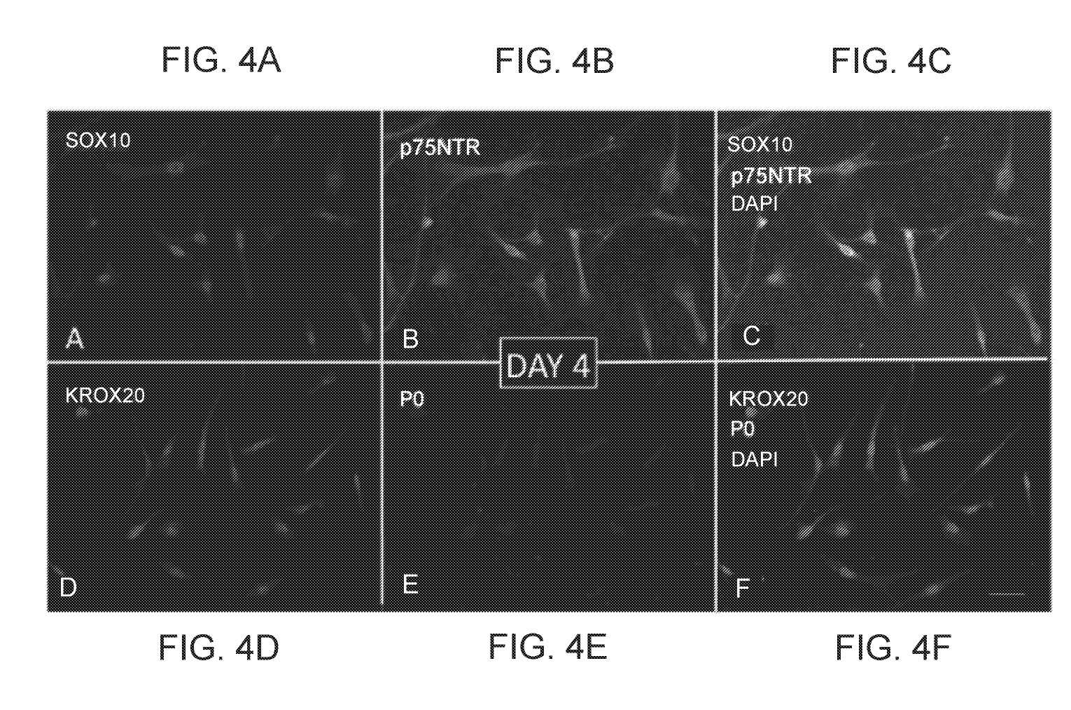

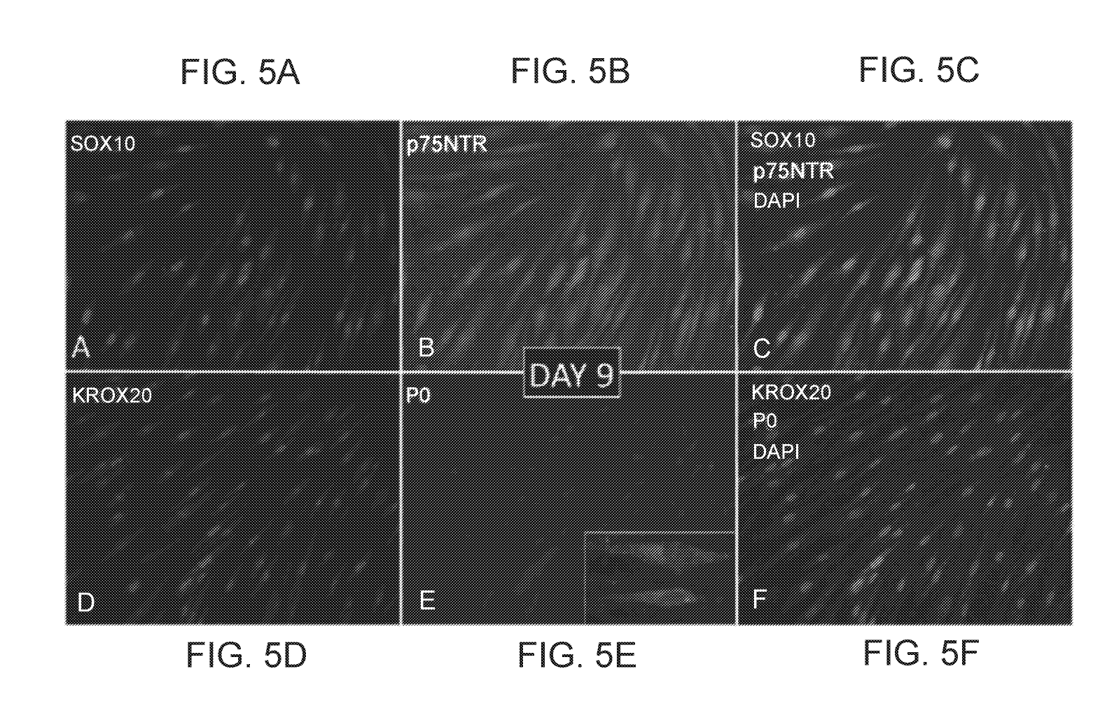

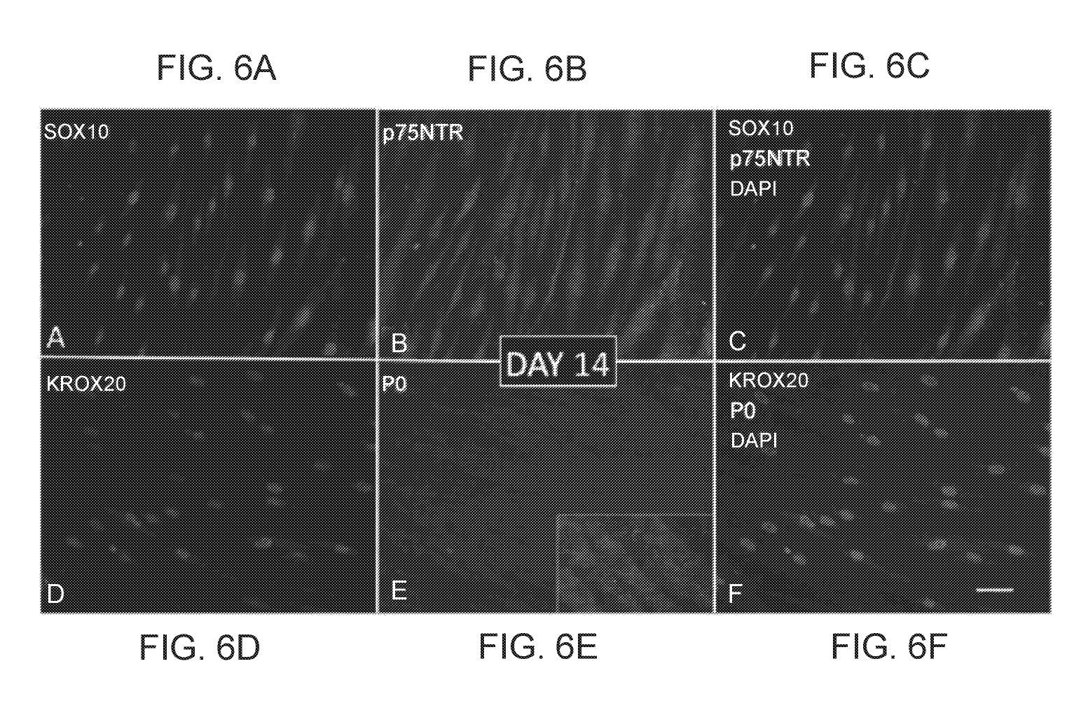

Nuclear immunoreactivity of SOX10 was observed in 52.5±8.1% of cells at the onset of differentiation on Day 0 (FIG. 3; Table 1). By Day 4, there was strong nuclear SOX10 immunopositivity in the majority of cells, 95.4±1.4%, which persisted until approximately Day 14 (89.0±2.5%) and subsequently became more prominent again in the cytoplasm (FIGS. 4-6; Table 1). KROX20 is a key Schwann cell marker that is regulated by SOX10 (Jessen and Mirksy, 2002; Reiprich et al, 2010).

KROX20 followed an expression pattern similar to that of SOX10 with the exception that nuclear localisation was maintained throughout the culture period. All cells expressed KROX20 in the cytoplasm. Nuclear expression of KROX20 was observed in 38.3±10.1% of cells on Day 0, in 74.7±5.6% on Day 4, in 91.9±0.8% on Day 9, in 95.6±1.2% of cells by Day 14, in 90.4±2.1% on Day 19 and in 90±2±2.1% of cells on Day 30 (FIGS. 3-6; Table 1).

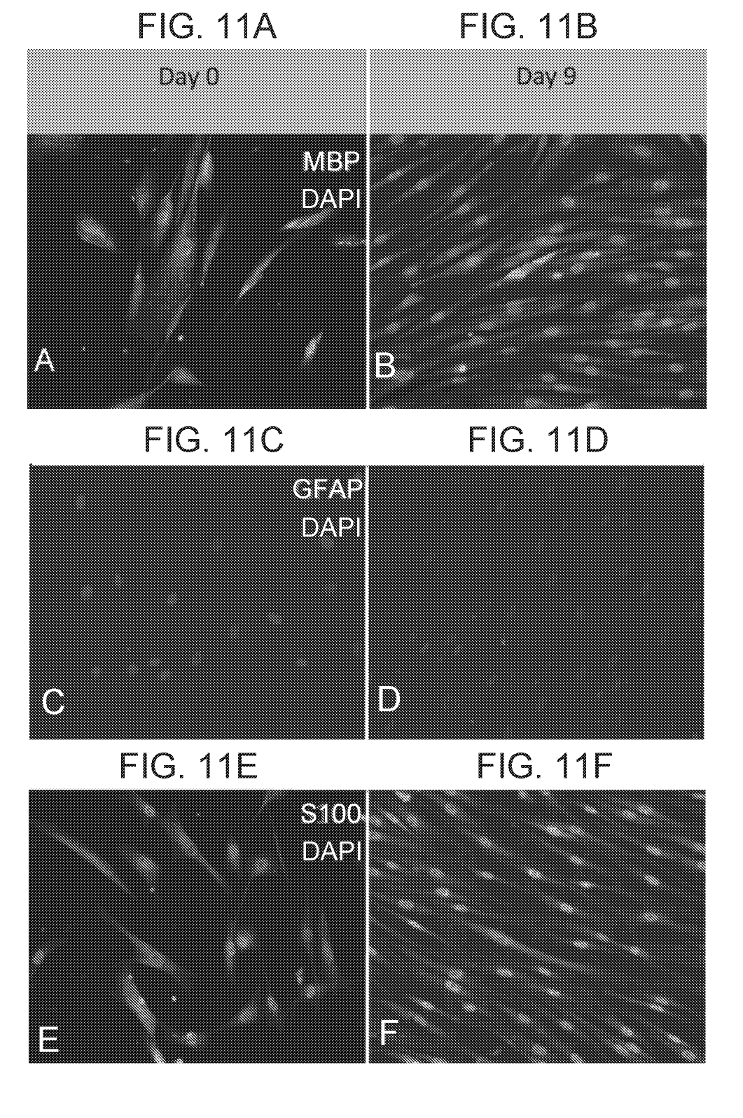

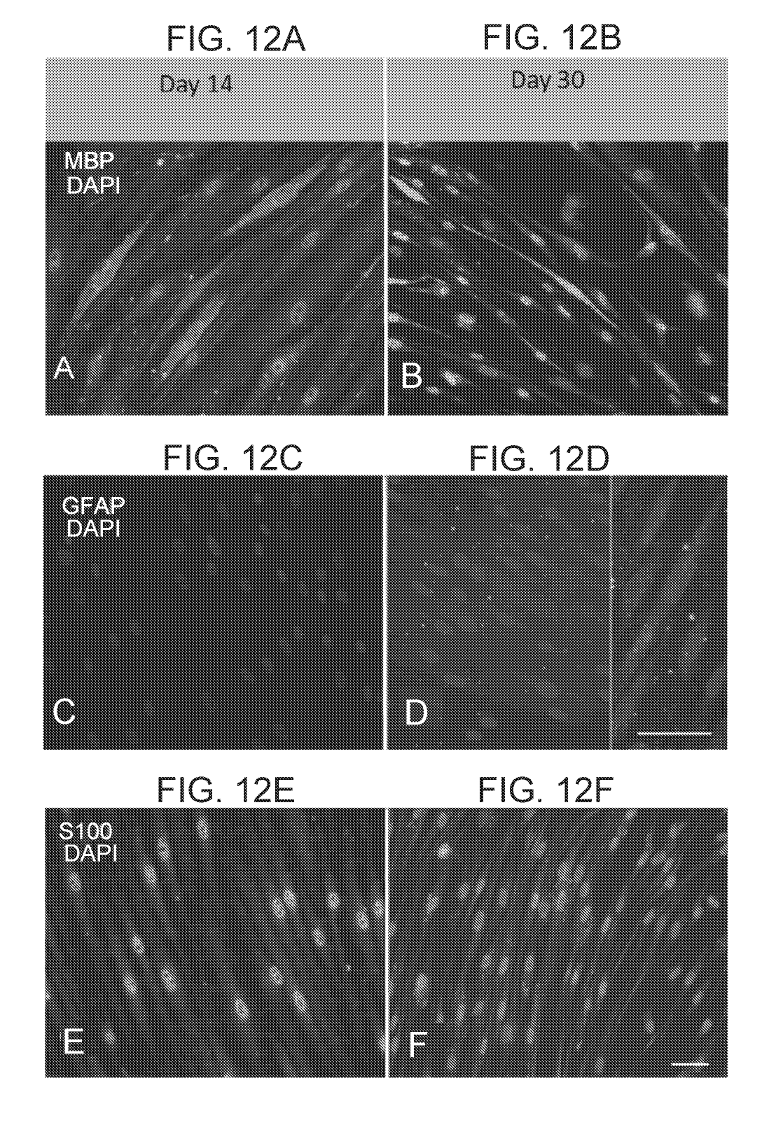

All cells expressed p75NTR (NGFR), myelin basic protein (MBP), and S100 immunoreactivity throughout the culture period (FIGS. 11-12). Intensity of p75NTR immunofluorescence decreased with progressing cell differentiation (FIGS. 3-6; FIGS. 11-12; Table 1).

In contrast, glial fibrillary acidic protein (GFAP) it immunoreactivity was not detected initially, and at low levels only after Day 14 of differentiation (Table 1), which is typical for myelinating Schwann cells. Cells were, however, intensely GFAP-immunoreactive in the absence of retinoic acid, SHH and CHIR99021, conditions under which SOX10 was expressed predominantly in the cytoplasm.

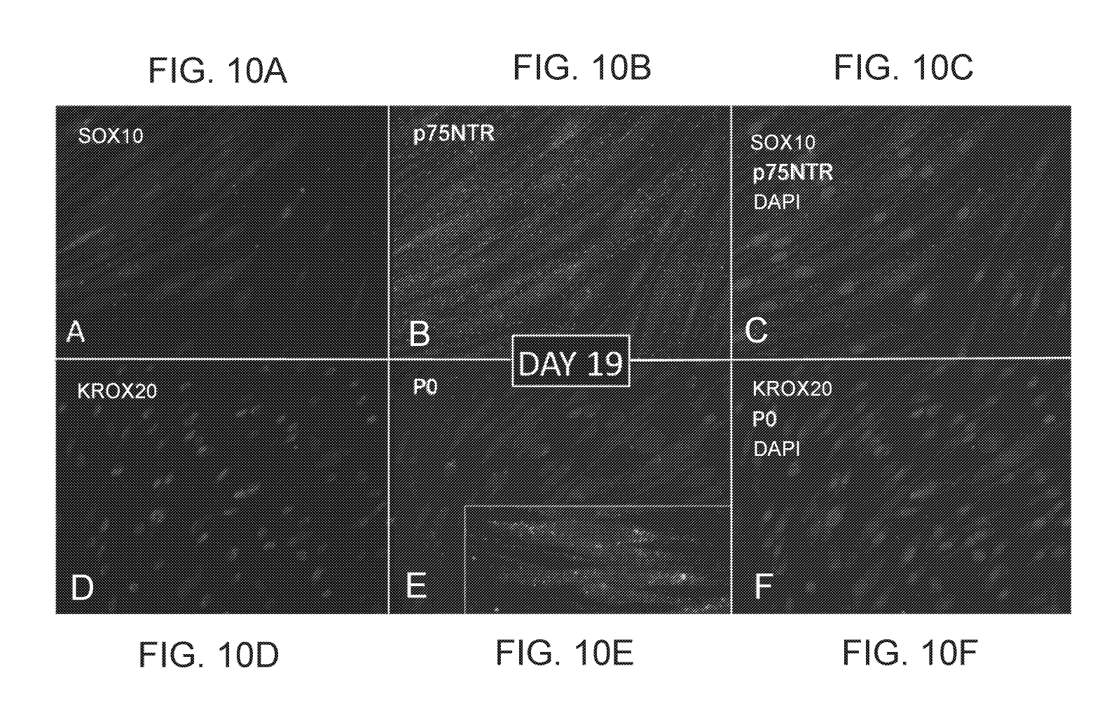

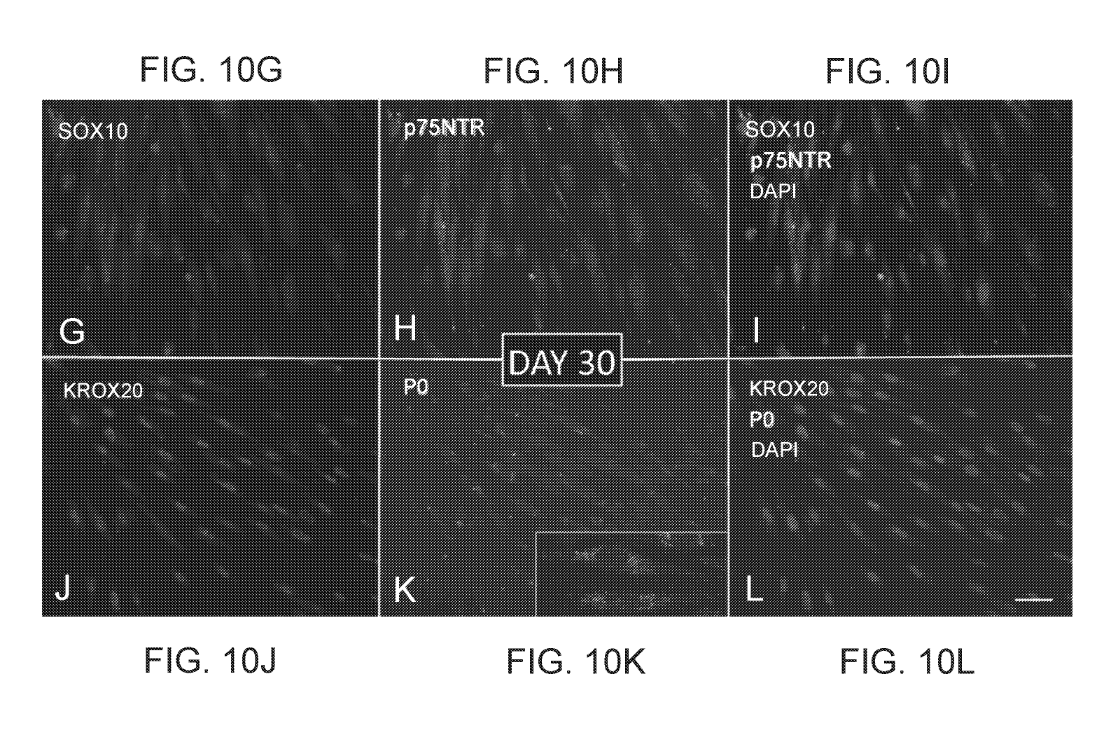

Initially there was no detectable P-zero (P0) immunoreactivity but P0 expression became apparent at low levels by differentiation Days 4-9, increased by Day 14, and remained strong throughout the remainder of the culture period (FIGS. 3-6; Table 1). FIG. 10A-10L show (cytoplasmic) SOX10, (reduced) p75NTR, (nuclear) KROX20 and (increased) P0 immunofluorescence on differentiation days 19 and 30.

An increase in the levels of SOX10, p75NTR and GFAP transcripts was observed by qPCR within 21 days after treatment began (Table 2A-2B).

TABLE 2A
qPCR analysis of expression of Schwann cell
markers before and after differentiation.

| | | Relative expression |
|---|---|---|
| | Day of | (Percent average ± |
| Gene | differentiation | SEM) |
| SOX10 | −3 | 100.0 ± 3.3 |
| | 21 | 505.0 ± 5.0* |
| p75NTR | −3 | 100.0 ± 3.9 |
| | 21 | 291.5 ± 5.7* |
| GFAP | −3 | 100.0 ± 9.7 |
| | 21 | 203.0 ± 4.4* |

Asterisk, statistically significant difference between pre-differentiation and post-differentiation. p<0.05 (n=3)

TABLE 2B
Summary of selected pertinent genes detected at higher
levels in hEPI-NCSC derived Schwann cells compared
to hEPI-NCSC in the Illumina expression profile.

| | Fold increase |
|---|---|
| Gene | (p < 0.001) |
| BDNF | 2.4 |
| CAV1 | 2.2 |
| EBP | 1.7 |
| FGF2 | 5.6 |
| FGF5 | 2.15 |
| IL-6 | 11.6 |
| KROX20 | 1.45 |
| MALL | 3.4 |
| MBP | 3.2 |
| OSBPL5 | 1.14 |
| SOD1 | 1.7 |

Two comprehensive gene expression profiles were obtained using the Illumina platform (see website at ncbi.nlm.nih.gov/geo/; GEO accession number GSE61273). One profile was made with RNA from undifferentiated hEPI-NCSC and the other with RNA from hEPI-NCSC derived Schwann cells. The Diff Score is a transformation of the p-value that provides directionality to the p-value based on the difference between the average signal in the reference group versus the comparison group. Of a total of 47,229 transcripts probed on the alumina HT12 Array, 3,690 genes were differentially expressed with a p-value≤0.01; of those, 2,954 genes were differentially expressed with a p-value≤0.001.

Transcripts for the Schwann cell-specific gene, KROX20, were up-regulated 1.45-fold (p<0.001) in Schwann cells. Highly differentially expressed genes concerned primarily metabolic processes, general differentiation markers, transporters of molecules, and genes regulating cell behaviour, as expected. In the Illumina profiles, 31 significantly down-regulated transcripts in Schwann cells (p<0.01) concerned cell migration, and an estimated greater than 200 down-regulated genes (p<0.01) regulate cell proliferation.

Extracellular proteases produced by migrating neural crest cells serve to degrade embryonic extracellular matrix. Four metalloproteases of the MMP family (MMP2, MMP3, IMP2 and BSG; p<0.0001), and 9 members of the ADAM family of proteinases (ADAM 12, 15, 17; ADAMTS 2, 5, 6; ADAMTSL 1, 4; and ADAMRSL4; p<0.001) were significantly down-regulated. Several myelin proteins were up-regulated.

Myelin basic protein (MBP) transcripts for instance were up-regulated 3.2-fold (p<0.001). Cholesterol is a major component of myelin. Twenty-one genes involved in cholesterol metabolism were up-regulated with high significance. For instance, MALL, a member of the MAL family of proteolipids that localises to cholesterol-enriched membranes was up-regulated 3.4-fold (p<0.001). Emopamil binding protein (EBP), up-regulated 1.7-fold (p<0.001), is a gene that encodes a sterol isomerase, which is involved in synthesizing 3β-hydroxysteroid-Δ8,Δ7-isomerase, which in turn is responsible for one of the final steps in the production of cholesterol. OSBPL5, a member of the oxysterol-binding protein (OSBP) family, a group of intracellular lipid receptors that play a key role in maintaining cholesterol balance in the body was up-regulated 1.14-fold (p<0.001). Transcripts for superoxide dismutase 1 (SOD1), mutations in which are implicated in multiple sclerosis, a de-myelinating disease, was up-regulated in Schwann cells 1.7 fold (p<0.001). Transcripts for the caveolin-1 (CAV1) gene, mutations in which are associated with Berardinelli-Seip congenital lipodystrophy, were up-regulated 2.2-fold (p<0.001).

In vivo, Schwann cells produce neurotrophins and other factors that support neuronal survival, including nerve growth factor (NGF; Rush, 1984), neurotrophin-4/5 (NT-4/5; Funakoshi et al, 1993), brain derived neurotrophic factor (BDNF; Meyer et al, 1992), fibroblast growth factor 2 (FGF2; Ornitz and Ito 2001), fibroblast growth factor 5 (FGF5; McGeachie et al, 2001), leukemia inhibitory factor (LIF; Kurek et al, 1996) and IL-6 (Bolin et al, 1995). NT-3, FGF2, BDNF, and LIF had a detection p-value of p=0 in both hEPI-NCSC and Schwann cells derived thereof; they are therefore expressed in both cell types with highest probability. FGFS was up-regulated 2.15-fold (p<0.001) in Schwann cells compared to hEPI-NCSC (detection p=0). BDNF was up-regulated 2.4-fold (p<0.0001), FGF2 5.7-fold (p<0.0001), and IL-6 11.6-fold (p<0.001). An important aspect for successful nerve regeneration includes angiogenesis. VEGFa and VEGFb had a detection p-value of p=0 in both hEPI-NCSC and in Schwann cells derived thereof; both genes are therefore expressed in both cell types with highest probability.

JUN (c-Jun) expression in Schwann cells is essential and sufficient for supporting nerve repair, as it regulates de-differentiation of myelinating Schwann cells into the regenerative type of Schwann cell (Arthur-Farraj et al, 2012). JUN gene transcripts were detected in both hEPI-NCSC and hEPI-NCSC derived Schwann cells with highest probability (detection p-value, p=0).

### EXAMPLE 5

### hEPI-NCSC Derived Schwann Cells Can Interact with Neurites

This Example describes experiments designed to illustrate the functionality of hEPI-NCSC derived Schwann cells.

Co-cultures of hEPI-NCSC derived Schwann cells and rodent dorsal root ganglia (DRG) explants were prepared by first treating the DRG explants with camptothecin to eliminate non-neuronal cells. Camptothecin efficiently removed endogenous Schwann cells but cytosine arabinoside did not (data not shown). However, camptothecin was selectively toxic to large diameter sensory neurons. Neuronal toxicity of camptothecin has been documented previously (Bhanu and Kondapi, 2010).

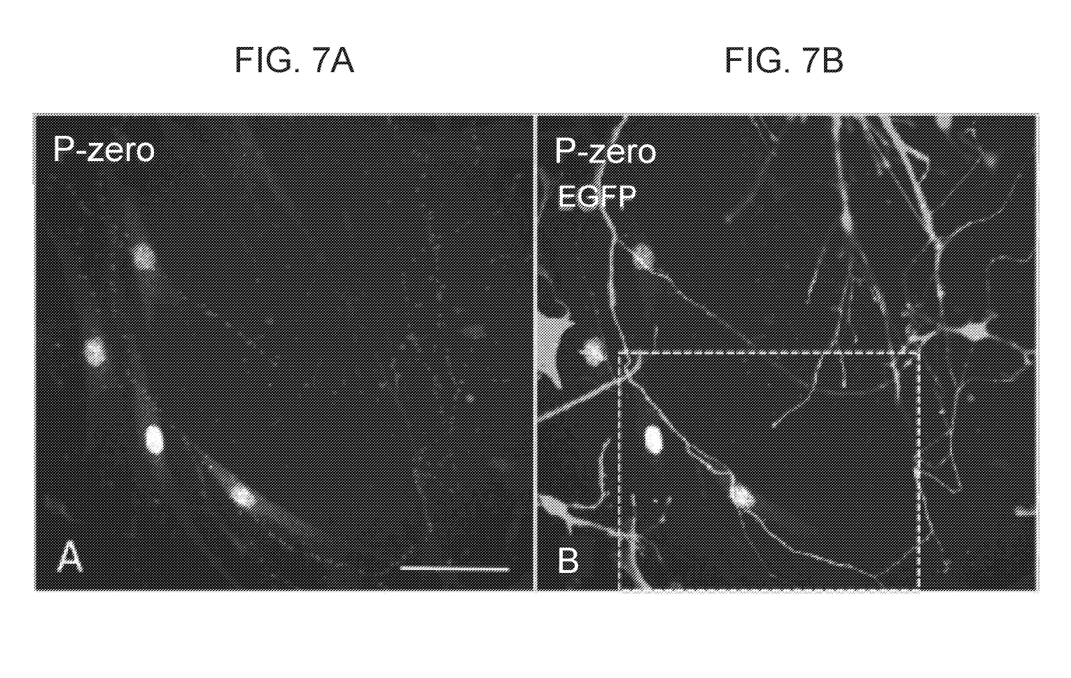

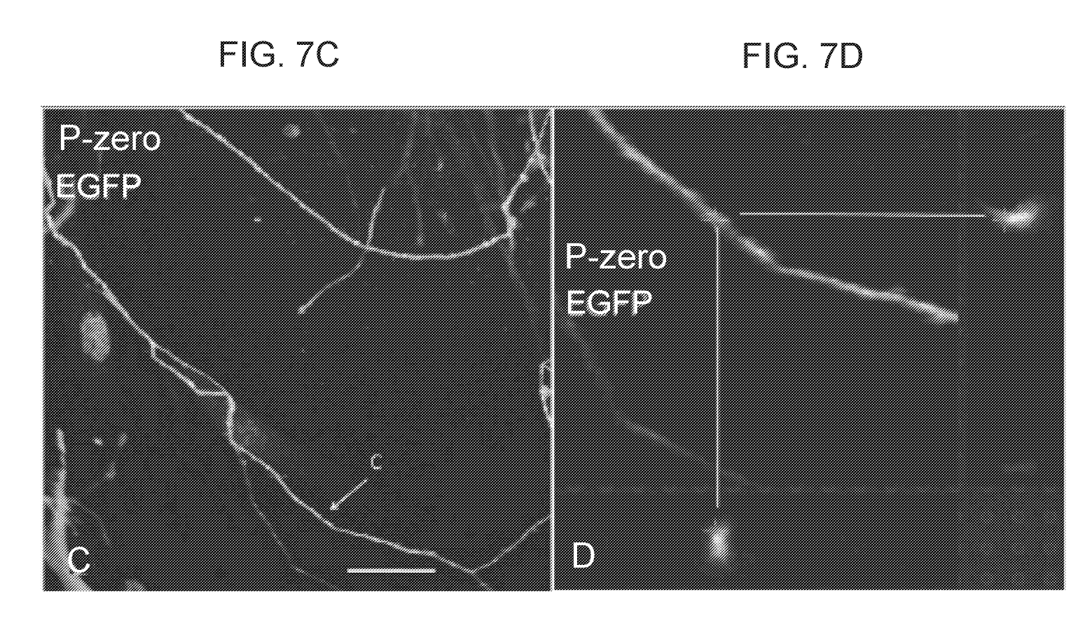

Confocal microscopy showed bead-like P-zero immunoreactivity above the human Schwann cells, which corresponded precisely with murine DRG neuronal processes (FIG. 74,-7B). Anti-human nuclear antibody immunofluorescence confirmed that all P-zero immunoreactive Schwann cells were of human origin (FIG. 7A-7C; pale blue fluorescence in original). FIG. 7D shows an orthogonal reconstruction of the area marked in FIG. 7C by an arrow, confirming close interaction of Schwann cell-derived P-zero immunoreactivity with neurite derived EGFP fluorescence. As it was difficult to remove all endogenous Schwann cells from adult mouse DRGs, and because E15.5 rat embryos do not yet have differentiated Schwann cells (Jessen et al, 1994), the co-culture experiments were repeated with whole DRG explants from rat embryos. While many non-neuronal cells emigrated in the absence of camptothecin, camptothecin treated DRG explains were clear of non-neuronal cells.

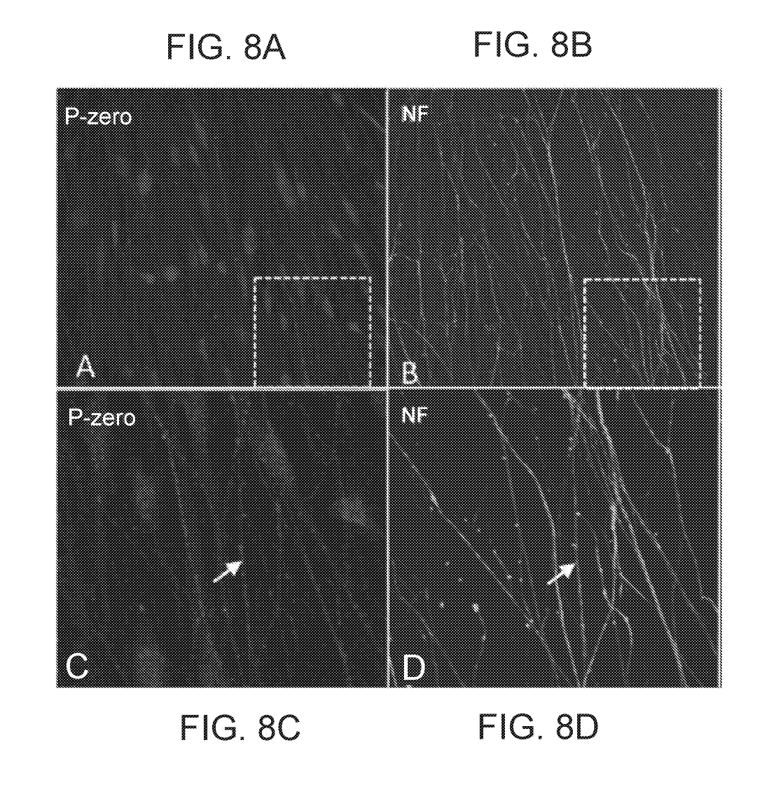

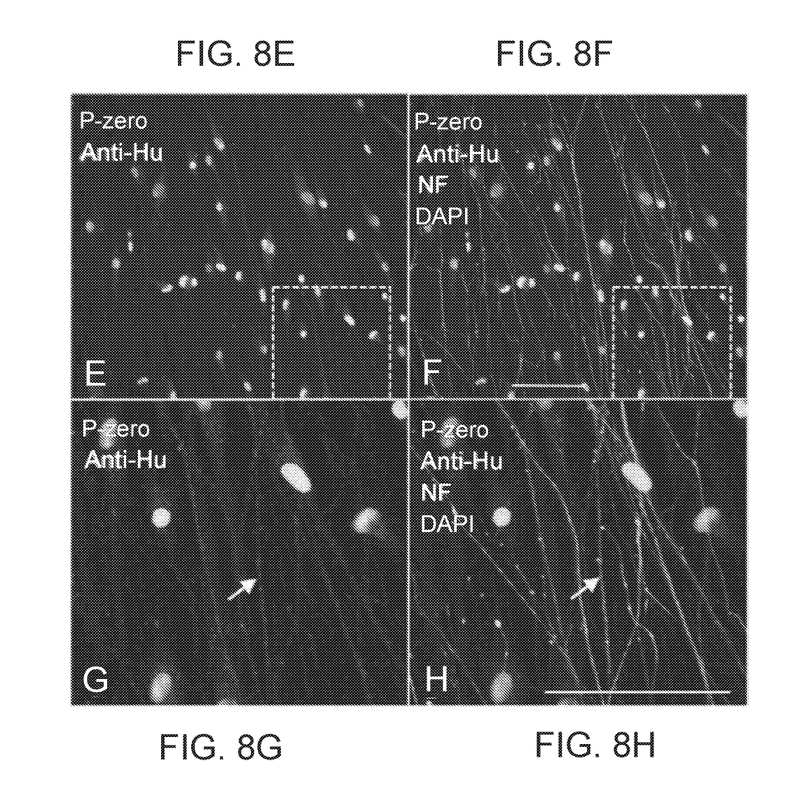

When hEPI-NCSC derived Schwann cells were added to the DRG explants, cells aligned with the neurites radiating from the explant (FIG. 8). Cell bodies and processes of Schwann cells were P-zero immunoreactive (FIG. 8A, 8C, 8E-8H). Again, bead-like Schwann cell P-zero immunoreactivity was consistently and precisely in close association with neurites (FIG. 8). All cells expressed anti-human nuclear antibody (FIG. 8E-8H) confirming the human origin of the Schwann cells. Anti-Hu/DAPI double stain showed that the co-cultures did not contain any non-human non-neural cells, as all DAPI positive nuclei were also anti-Hu immunoreactive (FIG. 8E-8H). Experiments in these two different co-culture systems showed close association of hEPI-NCSC derived Schwann cells with neurites.

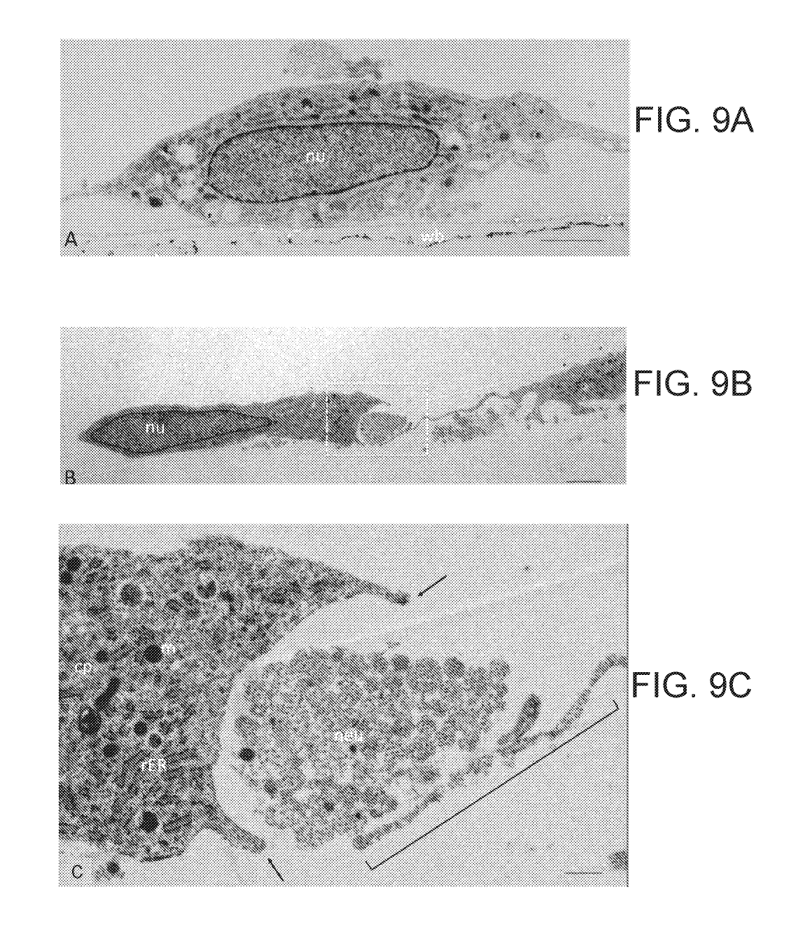

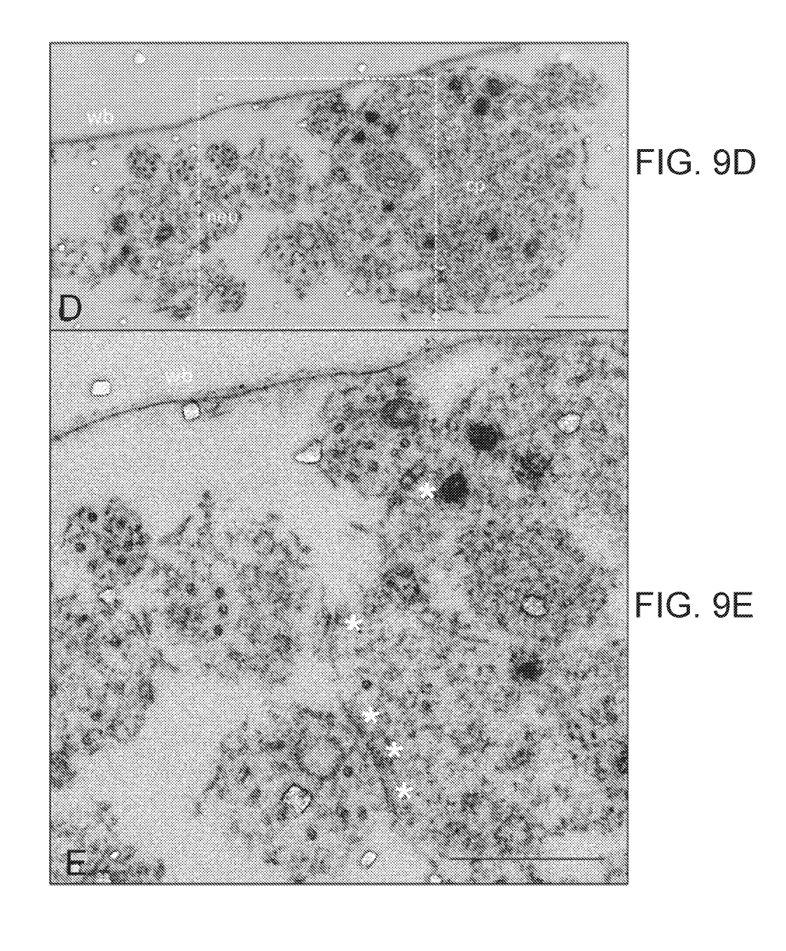

Next, interaction at the ultrastructural level was investigated in co-cultures of Schwann cells and rat embryo DRGs. FIG. 9 shows electron microscopy images of hEPI-NCSC DRG co-cultures. Schwann cells had the typical cellular and nuclear morphology (FIG. 9A). Bundles of DRG-derived neurites were engulfed by protrusions from Schwann cells, confirming the data obtained by confocal microscopy. The boxed area in FIG. 9B-9C) shows a cluster of neurites (neu) in cross section and in close apposition with Schwann cells. To the left of the bundle of axons is a Schwann cell that shows two protrusions towards the neurites. There is also a cellular protrusion (marked by a bracket) from the cell to the right that reaches towards the bundle of axons and which ends in its close proximity (FIG. 9C). Images in FIG. 9D-9E show a bundle of neurites (neu) in cross section to the left, and the cytoplasm (cp) of a cell to the right. Three neurites form close contacts with the membrane of the cell (5 white asterisks; FIG. 9E). Taken together, close interactions of hEPI-NCSC derived Schwann cells with rat embryo DRG axons were observed at the ultrastructural level also.

### REFERENCES

Allodi I, Udina E, Navarro X (2012). Specificity of peripheral nerve regeneration: interactions at the axon level. Prog Neurobiol. 98:16-37. Epub 2012 May 15.

Arthur-Farraj P J, Latouche M, Wilton D K Quint es S, Chabrol E, Banerjee A, Woodhoo A, Jenkins B, Rahman M, Turmaine M, Wicher G K, Mitter R, Greensmith L, Behrens A Raivich G, Mirsky R, Jessen K R. (2012) c-Jun reprograms Schwann cells of injured nerves to generate a repair cell essential for regeneration. Neuron. 75:633-647.

Bahnu, M U and Kondapi A K. (2010) Neurotoxic activity of a Topoisomerase-1 inhibitor, camptothecin, in cultured cerebellar granule neurons. NeuroToxicology 31,730-737

Banerjee A. Nürnberger S. Hennerbichler S. Riedl S, Schuh C M, Hacobian A, Teuschl A, Eibl J, Redl H, Wolbank S. (2014) In toto differentiation of human amniotic membrane towards the Schwann cell lineage. Cell Tissue Bank. 15:227-39. Epub 2013 Oct. 29.

Bolin L M, Verity A N, Silver J E, Shooter E M, Abrams J S. (1995) Interleukin-6 production by Schwann cells and induction in sciatic nerve injury. J Neurochem. 64:850-858.

Bunge M B. (2008) Novel combination strategies to repair the injured mammalian spinal cord. J Spinal Cord Med. 31(3):262-9.

Clewes O, Narytnyk A, Gillinder K R, Loughney A. D., Murdoch A. P., Sieber-Blum M. (2011). Human epidermal neural crest stem cells (hEPI-NCSC)-Characterization and directed differentiation into osteocyte and Melanocytes. Stem Cell Rev. Rep. 7:799-814.

Dezawa M, Takahashi I, Esaki M, Takeno M, Sawada H. (2001) Sciatic nerve regeneration in rats induced by transplantation of in vivo differentiated bone marrow-stromal cells. European J of Neurosci. 14: 1771-1776.

Evans G R. (2001) Peripheral nerve injury: a review and approach to tissue engineered constructs. Anat Rec. 263:396-404.

Funakoshi H, Frisen J, Barbany G, Timmusk T, Zachrisson O, Verge V M, Persson H. (1993). Differential expression of mRNAs for neurotrophins and their receptors after axotomy of the sciatic nerve. J Cell Biol. 123:455-465.

Gericota B, Anderson J S, Mitchell G, Borjesson D L, Sturges B K, Nolta J A, Sieber-Blum M. (2014). Canine epidermal neural crest stem cells: characterization and potential as therapy candidate for a large animal model of spinal cord injury. Stern Cells Transl Med. 3:334-45. Epub 2014 Jan. 17.

Griffin, M F Malahias, M, Hindocha S, Kahn W S. (2014). Peripheral Nerve Injury: Principles for Repair and Regeneration. The Open Orthopaedics Journal, 8, (Suppl 1: M10) 199-203.

Hu Y F, Gourab K, Wells C, Clewes O, Schmit BD, Sieber-Blum, M. (2010) Epidermal Neural Crest Stem Cell (EPI-NCSC)-Mediated Recovery of Sensory Function in a Mouse Model of Spinal Cord Injury. Stem Cell Rev. 6:186-198.

Hu Y F, Zhang Z J, Sieber-Blum M. (2006) An epidermal neural crest stern cell (EPI-NCSC) molecular signature. Stem Cells. 24:2692-702. Epub 2006 Aug. 24.

Jessen K R, Mirsky R. (2005). The origin and development of glial cells in peripheral nerves. Nat Rev Neurosci., 6:671-82.

Jessen K R, Brennan A, Morgan L, Mirsky R, Kent A, Hashimoto Y, Gavrilovic J. (1994). The Schwann Cell Precursor and Its Fate: A Study of Cell Death and Differentiation during Gliogenesis in Rat Embryonic Nerves. Neuron 12;509-527.

Jessen K R, Mirsky R. (1999). Schwann cells and their precursors emerge as major regulators of nerve development. Trends Neurosci. 22:402-10.

Jessen K R, Mirsky R. (2002) Signals that determine Schwann cell identity. J Anat. 200:367-76.

Jiang T M, Yang Z J, Kong C Z, Zhang H T. (2010) Schwann-like cells can be induction from human nestin-positive amniotic fluid mesenchymal stem cells. In Vitro Cell Dev Biol Anim. 46:793-800.

Johnson M, Bunge R, Wood P. (2001) Chapter eight, Primary cell cultures for the study of myelination. In Protocols for Neural Cell Culture, 95-115. Ed, Fedoroff S., Richardson A. Humana Press, Clifton, N.J., USA.

Kelsh R N. (2006) Sorting out Sox10 functions in neural crest development. Bioassays. 28:788-98.

Kingham P J, Kalbermatten D F, Mahay D, Armstrong S J, Wibera M, Terenghi G. (2007). Adipose-derived stem cells differentiate into a Schwann cell phenotype and promote neurite outgrowth in vitro. Exp Neurol, 207:267-274.

Kolar M K, Kingham P J. (2014) Regenerative effects of adipose-tissue-derived stem cells for treatment of peripheral nerve injuries. Biochem Soc Trans, 42:697-701.

Krause M P, Dworski S, Feinberg K, Jones K, Johnston A P, Paul S. Paris M, Peles E, Bagli D, Forrest C R, Kaplan D R, Miller F D. (2014) Direct genesis of functional rodent and human Schwann cells from skin mesenchymal precursors. Stem Cell Reports. 19:85-100.

Kurek J B, Nouri S, Kannourakis G, Murphy M, Austin L. (1996) Leukemia inhibitory factor and interleukin-6 are produced by diseased and regenerating skeletal muscle. Muscle Nerve. 19:1291-301.

Laping N J, Grygielko E, Mathur A, Butter S. Bomberger J, Tweed C, Martin W. Fomwald J, Lehr R, Harling J, Gaster L, Callahan J F, Olson B A. (2002) Inhibition of transforming growth factor (TGF)-betal-induced extracellular matrix with a novel inhibitor of the TGF-beta type I receptor kinase activity: SB-431542. Mol Pharmacol. 62(1):58-64.

Le Douarin N M, Kalcheim C. (1999). ‘The Neural Crest’. New York: Cambridge University Press.

Lee J H, Chung W H, Kang E H, Chung D J, Choi C B, Chang H S, Lee J H, Hwang S H, Han H, Choe B Y, Kim H Y. (2011) Schwann cell-like remyelination following transplantation of human umbilical cord blood (hUCB)-derived mesenchymal stem cells in dogs with acute spinal cord injury. J Neurol Sci. 300:86-96.

Liu Z, Gao W, Wang Y, Zhang W, Liu H, Li Z. (2011) Neuregulin-1β regulates outgrowth of neurites and migration of neurofilament 200 neurons from dorsal root ganglial explants in vitro. Peptides 32:1244-1248.

Malin S., Davis B., Molliver D. (2007) Production of dissociated sensory neuron cultures and considerations for their use in studying neuronal function and plasticity. Nature Protocols 2:152-160.

McGeachie A B, Koishi K, Imamura T, McLennan I S, (2001) Fibroblast growth factor-5 is expressed in Schwann cells and is not essential for motoneuron survival. Neuroscience. 104:891-899.

McMahill B G. Borjesson D L, Sieber-Blum M, Nolta J A, Sturges B K. (2014) Stem Cells in Canine Spinal Cord Injury—Promise for Regenerative Therapy in a Large Animal Model of Human Disease. Stem Cell Rev. August 31. [Epub ahead of print]

Meyer M, Matsuoka I, Wetmore C, Olson L, Thoenen H. (1992) Enhanced synthesis of brain-derived neurotrophic factor in the lesioned peripheral nerve: different mechanisms are responsible for the regulation of BDNF and NGF mRNA. J Cell Biol. 119:45-54.

Morgan L, Jessen K R, Mirsky R. (1994) Negative regulation of the Po gene in Schwann cells: suppression of Po mRNA and protein induction in cultured Schwann cells by FGF2 and TGFb1, TGFb2 and TGFb3. Development 120:1399-1409.

McNeill B, Perez-Iratxeta C, Mazerolle C, Furimsky M, Mishina Y, Andrade-Navarro M A, Wallace V A. (2012) Comparative genomics identification of a novel set of temporally regulated hedgehog target genes in the retina. Mol Cell Neurosci. 49:333-40.

Narytnyk A, Gillinder K, Verdon B, Clewes O, Sieber-Blum M. (2014a) Neural crest stem cell-specific deletion of Pygopus2 gene modulates hair follicle development. Stem Cell Rev. 10:60-68.

Narytnyk A, Verdon B, Loughney A, Sweeney M, Clewes O, Taggart M J, Sieber-Blum M. (2014b) Differentiation of human epidermal neural crest stern cells (hEPI-NCSC) into virtually homogenous populations of dopaminergic neurons. Stem Cell Rev. 10:316-326.

Noble J. Munro C A, Prasad V S, Prasad V S, Midha R. (1998) Analysis of upper and lower extremity peripheral nerve injuries in a population of patients with multiple injuries. J Trauma. 45:116-22.

Okabe, M, Ikawa, M. Kominami, K, Nakanishi, T., Nishimune, Y. (1997) ‘Green mice’ as a source of ubiquitous green cells. FEBS Lett. 407:313-319.

Omitz D M, Itoh N. (2001) Fibroblast growth factors. Genome Biol.2: reviews3005.1-reviews3005.12.

Päiväläinen S, Nissinen M, Honkanen H, Lahti O., Kangas, S. M., Peltonen J., Peltonen S., Heape A. M. (2008) Myelination in mouse dorsal root ganglion/Schwann cell cocultures. Mol. Cell. Neurosci. 37:568-578.

Pan Y, Cai S. (2012) Current state of the development of mesenchymal stem cells into clinically applicable Schwann cell transplants. Mol Cell Biochem. 368:127-35.

Pfister B J, Gordon T, Loverde J R, Kochar A S, Mackinnon S E, Cullen D K. (2011) Biomedical engineering strategies for peripheral nerve repair: surgical applications, state of the art, and future challenges. Crit Rev Biomed Eng. 39:81-124.

Razavi S, Mardani M, Kazemi M, Esfandiari E, Narimant M, Esmaeili A, Ahmadi N. (2013) Effect of leukemia inhibitory factor on the myelinogenic ability of Schwann-like cells induced from human adipose-derived stem cells. Cell Mol Neurobiol. 33:283-9.

Richardson M K, Sieber-Blum M. (1993) Pluripotent neural crest cells in the developing skin of the quail embryo. Dev Biol. 157:348-358.

Rehberg S, Lischka P, Glaser G, Stamminger T, Wegner M, Rosorius O. (2002) Sox10 is an active nucleocytoplasmic shuttle protein, and shuttling is crucial for Sox10-mediated transactivation. Mol Cell Biol. 22:5826-5834.

Reid A J, Sun M, Wiberg M, Downes S, Terenghi G, Kingham P J. (2011) Nerve repair with adipose-derived stem cells protects dorsal root ganglia neurons from apoptosis. Neuroscience, 199:515-522.

Reiprich S, Kriesch J, Schreiner S, Wegner M. (2010) Activation of Krox20 gene expression by Sox10 in myelinating Schwann cells. J Neurochem. 112:744-54.

Ren Y J, Zhang S, Mi R, Liu Q, Zeng X, Rao M, Hoke A, Mao H Q. (2013) Enhanced differentiation of human neural crest stem cells towards the Schwann cell lineage by aligned electrospun fiber matrix. Acta Biomater. 9:7727-36.

Rosberg H E, Carlsson K S, Höjgärd S, Lindgren B, Lundborg G, Dahlin L B. (2005) Injury to the human median and ulnar nerves in the forearm—analysis of costs for treatment and rehabilitation of 69 patients in southern Sweden. J Hand Surg Br. 30:35-39.

Rush R A. (1984) Immunohistochemical localization of endogenous nerve growth factor. Nature. 312(5992):364-7.

Shirosaki Y, Hayakawa S, Osaka A, Lopes M A, Santos J D, Geuna S, Mauricio A C. (2014). Challenges for nerve repair using chitosan-siloxane hybrid porous scaffolds. Biomed Res Int. 2014:153808.

Sieber-Blum M, Grim M, Hu Y F, Szeder V. (2004) Pluripotent neural crest stem cells in the adult hair follicle. Dev Den. 231:258-69.

Sieber-Blum M, Grim M. (2004) The adult hair follicle: cradle for pluripotent neural crest stem cells. Birth Defects Res C Embryo Today. 72:162-72.

Sieber-Blum M. (2014) Human epidermal neural crest stem cells as candidates for cell-based therapies, disease modeling, and drug discovery. Birth Defects Res C Embryo Today. 102:221-226.

Sieber-Blum M, Schnell L, Grim M, Hu Y F, Schneider R, Schwab M E. (2006) Characterization of epidermal neural crest stem cell (EPI-NCSC) grafts in the lesioned spinal cord. Mol Cell Neurosci. 32:67-81.

Tomita K, Madura T, Sakai Y, Yano K, Terenghi G, Hosokawa K. (2013) Glial differentiation of human adipose-derived stem cells: implications for cell-based transplantation therapy. Neuroscience. 236:55-65.

Werner T, Hammer A, Wahlbuhl M, Bösl M R, Wegner M. (2007) Multiple conserved regulatory elements with overlapping functions determine Sox10 expression in mouse embryogenesis. Nucleic Acids Res. 35:6526-38.

Wiliam R R, Bunge M B. (2012) Schwann cell transplantation: a repair strategy for spinal cord injury? Prot Brain Res. 201:295-312. doi: 10.1016/B978-0-444-59544-7.00014-7.

Ziegler L. Grigoryan S, Yang I H, Thakor N V, Goldstein R S. (2011) Efficient generation of schwann cells from human embryonic stem cell-derived neurospheres. Stem Cell Rev. 7:394-403.

All patents and publications referenced or mentioned herein are indicative of the levels of skills of those skilled in the art to which the invention pertains, and each referenced patent or publication is hereby specifically incorporated herein by reference in its entirety. Applicants reserve the right to physically incorporate any and all materials, text, and information from any such cited patents or publications into this specification.

The following statements summarize and describe aspects of the invention.

### Statements Summarizing Aspects of the Invention:

1. A method of pre-treating one or more epidermal neural crest stem cells (EPI-NCSC) for differentiation into Schwann cells, comprising:
   a. contacting one or more epidermal neural crest stern cells (EPI-NCSC) with a pre-treatment culture medium comprising sonic hedgehog and a WNT signaling activator for at least 1 day to generate pre-treated epidermal neural crest stem cells; and
   b. incubating one or more of the pre-treated epidermal neural crest stern cells in a culture media without comprising sonic hedgehog and a WNT signaling activator for at least one day, to thereby generate a population of pre-treated epidermal neural crest stem cells.
2. The method of statement 1, wherein the epidermal neural crest stem cells are obtained from one or more hair follicle bulge.
3. The method of statement 1 or 2, wherein the epidermal neural crest stem cells are not skin derived precursors.
4. The method of any of statements 1-3, wherein the epidermal neural crest stem cells are an allogenic or autologous cells from a mammalian subject who may be in need of Schwann cells.
5. The method of any of statements 1-4, wherein the one or more epidermal neural crest stem cells are cultured in the pre-treatment culture medium for about 1 to about 4 days.
6. The method of any of statements 1-5, wherein the one or more epidermal neural crest stem cells are cultured in the pre-treatment culture medium for 2 days.
7. The method of any of statements 1-6, wherein the sonic hedgehog and/or the WNT signaling activator are in the pre-treatment culture medium in an amount sufficient to increase nuclear localization of SOX10 expression in the epidermal neural crest stem cells.
8. The method of any of statements 1-7, wherein the sonic hedgehog and/or the WNT signaling activator are in the pre-treatment culture medium in an amount sufficient to increase KROX20 expression in the epidermal neural crest stern cells.
9. The method of any of statements 1-8, wherein the pre-treatment culture medium contains about 50 ng/ml to about 2 mg/ml, or about 100 ng/ml to about 1.5 mg/ml, or about 200 ng/ml to about 1 mg/ml sonic hedgehog, or about 500 ng/ml sonic hedgehog.
10. The method of any of statements 1-9, wherein the pre-treatment culture medium contains about 1.0 nanomolar to about 2 micromolar, or about 10.0 nanomolar to about 1.0 micromolar, or about 100 nanomolar to about 800 nanomolar, or at about 200 nM to about 700 nM, or at about 500 nanomolar of the WNT signaling activator.
11. The method of any of statements 1-10, wherein the WNT signaling activator is CHIR99021 (6-(2-(4-(2,4-dichlorophenyl)-5-(4-methyl-1H-imidazol-2-yl)pyrimidin-2-ylamino)ethylamino)nicotinonitrile); 1-azakenpaullone (9-Bromo-7,12-dihydro-pyrido[3′,2′:2,3]azepino[4,5-b]indol-6(5H)-one), BIO ((2′Z,3′E)-6-Bromoindirubin-3′-oxime); AR-A014418 (N-(4-Methoxybenzyl)-N′-(5-nitro-1,3-thiazol-2-yl)urea); Indirubin-3′-monoxime; 5-Iodo-indirubin-3′-monoxime; kenpaullone (9-Bromo-7,12-dihydroindolo-[3,2-d] [1]benzazepin-6(5H)-one); SB-415286 (3-[(3-Chloro-4-hydroxyphenyl)amino]-4-(2-nitro-phenyl)-1H-pyrrole-2,5-dione); SB-216763 (3-(2,4-Dichlorophenyl)-4-(1-methyl-1H-indol-3-yl)-1H-pyrrole-2,5-dione); Maybridge SEW00923SC (2-anilino-5-phenyl-1,3,4-oxadiazole); (Z)-5-(2,3-Methylenedioxyphenyl)-imidazolidine-2,4-dione; TWS119 (3-(6-(3-aminophenyl)-7H-pyrrolo[2,3-d]pyrimidin-4-yloxy)phenol); CHIR98014 (N2-(2-(4-(2,4-dichlorophenyl)-5-(1H-imidazol-1-yl)pyrimidin-2-ylamino)ethyl)-5-nitropyridine-2,6-diamine); SB415286 (3-(3-chloro-4-hydroxyphenylamino)-4-(2-nitrophenyl)-1H-pyrrole-2,5-dione); Tideglusib (2-(1-naphthalenyl)-4-(phenylmethyl)); LY2090314 (3-imidazo[1,2-a]pyridin-3-yl-4-[1,2,3,4-tetrahydro-2-(1-piperidinylcarbonyl)-pyrrolo[3,2,1-jk][1,4]benzodiazepin-7-yl]); lithium salt; or any combination thereof.
12. The method of any of statements 1-11, wherein the WNT signaling activator is CHIR99021.
13. The method of any of statements 1-12, wherein P-zero (P0) and glial fibrillary acidic protein (GFAP) immunoreactivity is not detectable in EPI-NCSC after pre-treatment.
14. A method of generating Schwann cells comprising:
   a. contacting one or more epidermal neural crest stem cells (EPI-NCSC) with a first differentiation culture medium comprising a reducing agent, TGFβ1 inhibitor, and a first amount of serum for 1-3 days to generate a first cell population;
   b. contacting the first cell population with a second differentiation culture medium comprising an RARγ agonist, the TGFβ1 inhibitor, and the first amount of serum for 1-4 days to generate a second cell population; and
   c. contacting the second cell population with a third differentiation culture medium comprising the RARγ agonist, the TGFβ1 inhibitor, a second amount of serum, a fibroblast growth factor, a platelet derived growth factor, a neuregulin, and an agent that elevates cAMP levels for about 4 to about 28 days to thereby generate EPI-NCSC-derived Schwann cells.
15. The method of statement 14, wherein the epidermal neural crest stem cells are pre-treated prior to contact with the first differentiation culture medium in a method comprising:
   a. contacting one or more epidermal neural crest stem cells (EPI-NCSC) with a pre-treatment culture medium comprising sonic hedgehog and a WNT signaling activator for at least 1 day to generate pre-treated epidermal neural crest stem cells; and
   b. incubating one or more of the pre-treated epidermal neural crest stem cells in a culture media without comprising sonic hedgehog and a WNT signaling activator for at least one day, to thereby generate a population of pre-treated epidermal neural crest stem cells.
16. The method of statement 14 or 15, wherein the epidermal neural crest stem cells are not skin derived precursors.
17. The method of any of statements 14-16, wherein the epidermal neural crest stem cells are an allogenic or autologous cells from a mammalian subject who may be in need of Schwann cells.
18. The method of any of statements 14-17, wherein the EPI-NCSC-derived Schwann cells express KROX20, SOX10, P-zero (P0), MBP, or a combination thereof.
19. The method of any of statements 14-18, wherein the EPI-NCSC-derived Schwann cells express KROX20, MBP, P-zero (P0), S100, but do not express GFAP.
20. The method of any of statements 14-19, wherein the EPI-NCSC-derived Schwann cells express KROX20, MBP, P-zero (P0), S100, and low levels of GFAP.
21. The method of any of statements 14-20, wherein the EPI-NCSC-derived Schwann cells express low levels of p75NTR.
22. The method of any of statements 14-21, wherein the EPI-NCSC-derived Schwann cells express SOX10, KROX20, or a combination thereof in cell nuclei.
23. The method of any of statements 14-22, wherein the first amount of serum is at a concentration of about 5% to about 20%, or about 10%.
24. The method of any of statements 14-23, wherein the serum is fetal bovine serum.
25. The method of any of statements 14-24, wherein the reducing agent in the first differentiation culture medium is a disulphide bond reducing agent.
26. The method of any of statements 14-25, wherein the reducing agent is dithiothreitol (DTT), β-mercaptoethanol ((βME), or a mixture thereof.
27. The method of any of statements 14-26, wherein reducing agent is β-mercaptoethanol.
28. The method of any of statements 14-27, wherein the reducing agent is present in the first differentiation culture medium at a concentration of about 0.1 millimolar to about 10 millimolar, or about 0.2 millimolar to about 5 millimolar, or about 0.5 millimolar to about 1.5 millimolar, or about 1 millimolar.
29. The method of any of statements 14-28, wherein the TGFβ inhibitor is 4-[4-(1,3-benzodioxl-5-yl)-5-(2-pyridinyl)-1H-imidazol-2-yl]benzamide (SB 431542); 3-(6-Methyl-2-pyridinyl)-N-phenyl-4-(4-quinolinyl)-1H-pyrazole-1-carbothioamide (A83-01); 2-(3-(6-Methylpyridine-2-yl)-1H-pyrazol-4-yl)-1,5-naphthyridine (SJN 2511); 4-[4-(2,3-Dihydro-1,4-benzodioxin-6-yl)-5-(2-pyridinyl)-1H-imidazol-2-yl]benzamide (D 4476); 4-[3-(2-Pyridinyl)-1H-pyrazol-4-yl]-quinoline (LY 364947); 2-(4-(benzo[d][1,3]dioxol-5-yl)-2-tert-butyl-1H-imidazol-5-yl)-6-methylpyridine (SB505124); 6-[2-(1,1-Dimethylethyl)-5-(6-methyl-2-pyridinyl)-1H-imidazol-4-yl]quinoxaline (SB 525334); 2-(5-Chloro-2-fluorophenyl)-4-[(4-pyridyl)amino]pteridine (SD 208); 4-(6-(4-(piperazin-1-yl)phenyfl)pyrazolo[1,5-a]pyrimidin-3-yl)quinoline (LDN-193189 or any combination thereof.
30. The method of any of statements 14-29, wherein the TGFβ1 inhibitor is SB431542.
31. The method of any of statements 14-30, wherein the TGFβ1 inhibitor is at a concentration sufficient to inhibit the cells from differentiating into neurons.

32. The method of any of statements 14-31, wherein the TGFβ1 inhibitor is at a concentration of about 0.5 micromolar to about 30 micromolar, or about 1.0 micromolar to about 20 micromolar, or about 3 micromolar to about 15 micromolar, or about 10 micromolar in a solution.
33. The method of any of statements 14-32, wherein the RAR γ agonist is CD1530; CD666; NRX204647; retinoic acid; all-trans retinoic acid (ATRA); 9-cis retinoic acid; all-trans 3-4 didehydro retinoic acid; 4-oxo retinoic acid; Retinol; 4-[(E)-2-(5,6,7,8-tetrahydro-5,5,8,8-tetramethyl-2-naphthalenyl)-1-propenyl]benzoic acid; 4-(5-methoxymethyl-5-methyl-2,3,4,5-tetrahydro-1-benzo[b]oxepin-8-yl-ethynyl)-benzoic acid; 4-(5-ethoxymethyl-5-methyl-2,3,4,5-tetrahydrobenzo[b]oxepin-8-ylethynyl)-benzoic acid; 4-(5-methyl-5-propoxymethyl-2,3,4,5-tetrahydrobenzo[b]oxepin-8-ylethynyl)-benzoic acid; (E)-4-[2-(5-methoxymethyl-5-propyl-2,3,4,5-tetrahydrobenzo[b]oxepin-8-yl)-vinyl]-benzoic acid; (E)-4-[2-(5-methoxymethyl-5-methyl-2,3,4,5-tetrahydro-benzo[b]oxepin-8-yl)-vinyl]-benzoic acid; (E)-4-[2-(5-methyl-5-propoxymethyl-2,3,4,5-tetrahydrobenzo[b]oxepin-8-yl)-vinyl]-benzoic acid; 4-(5-methoxymethyl-5-methyl-2,3,4,5-tetrahydrobenzo[b]thiepin-8-ylethynyl)-benzoic acid; 4-(5-ethoxymethyl-5-methyl-2,3,4,5-tetrahydrobenzo[b]thiepin-8-ylethynyl)-benzoic acid; (E)-4-[2-(5-ethoxymethyl-5-methyl-2,3,4,5-tetrahydrobenzo[b]thiepin-8-yl)-vinyl]-benzoic acid; (E)-4-[2-(5-methoxymethyl-5-propyl-2,3,4,5-tetrahydrobenzo[b]thiepin-8-yl)-vinyl]-benzoic acid; 4-(4-methoxymethyl-4-methyl-chroman-6-ylethynyl)-benzoic acid; (E)-4-[2-(4-methoxymethl-4-methyl-chroman-6-yl)-vinyl]-benzoic acid; or any combination thereof.
34. The method of any of statements 14-33, wherein the RAR γ agonist is all-trans retinoic acid.
35. The method of any of statements 14-34, wherein RARγ agonist is at a concentration of about 5 ng/ml to about 60 ng/ml, or about 20 ng/ml to about 50 ng/ml, or about 35 ng/ml.
36. The method of any of statements 14-35, wherein RARγ agonist is present in the third differentiation medium for longer than 5 days, or longer than 7 days, or longer than 9 days.
37. The method of any of statements 14-36, wherein the serum in the third differentiation culture medium is fetal bovine serum.

38. The method of any of statements 14-37, wherein the second amount of serum present in the third differentiation culture medium is less than the first amount of serum. 39. The method of any of statements 14-38, wherein the second amount of serum present in the third differentiation culture medium is about 0.1% to about 4%, or about 0.3 to about 3%, or about 0.5% to about 1.5%, or about 1.0%.
40. The method of any of statements 14-39, wherein the RARγ agonist and the TGFβ1 inhibitor are present in the third differentiation culture medium at the same concentrations as in the second differentiation culture media.
41. The method of any of statements 14-40, wherein the fibroblast growth factor is basic fibroblast growth factor or FGF2.
42. The method of any of statements 14-41, wherein the fibroblast growth factor is present in the third differentiation culture medium at a concentration of about 1 ng/ml to about 30 ng/ml, or at about 3 ng/ml to about 20 ng/ml, or at about 5 ng/ml to about 15 ng/ml, or about 10 ng/ml.
43. The method of any of statements 14-42, wherein the platelet derived growth factor (PDGF) is PDGF-B, PDGF-BB, PDGF-AB, PDGF-C, PDGF-D, PDGF-CC, PDGF-DD, PDGF-BC, PDGF-AC, PDGF-AD, PDGF-BD or a combination thereof.
44. The method of any of statements 14-43, wherein the platelet derived growth factor is PDGF-BB.
45. The method of any of statements 14-44, wherein the PDGF is present in the third differentiation culture medium at a concentration of about 0.5 ng/ml to about 25 ng/ml, or at about 1 ng/ml to about 15 ng/ml, or at about 2 ng/ml to about 10 ng/ml, or at about 5 ng/ml.
46. The method of any of statements 14-45, wherein the agent that elevates cAMP levels is forskolin, cholera toxin, caffeine, theophylline, buciadesine (dibutyryl cAMP, db cAMP), pertussis toxin, or a combination thereof.
47. The method of any of statements 14-46, wherein the adenylyl cyclase is present in the third differentiation culture medium at a concentration of about 0,5 μM to about 25 μM, or at about 1 μM to about 15 μM, or at about 2 μM to about 10 μM, or at about 5 μM.
48. The method of any of statements 14-47, wherein the neuregulin-1 is present in the third differentiation culture medium at a concentration of about 50 ng/ml to about 500 ng/ml, or at about 100 ng/ml to about 400 ng/ml, or at about 150 ng/ml to about 300 ng/ml, or at about 200 ng/ml.
49. The method of any of statements 14-48, wherein the sonic hedgehog and/or the signaling activator are in the pre-treatment culture medium in an amount sufficient to increase nuclear SOX10 expression in the epidermal neural crest stem cells.
50. The method of any of statements 14-49, wherein the sonic hedgehog and/or the WNT signaling activator are in the pre-treatment culture medium in an amount sufficient to increase KROX20 expression in the epidermal neural crest stern cells.
51. The method of any of statements 14-50, wherein the pre-treatment culture medium contains about 50 ng/m1 to about 2 mg/ml, or about 100 ng/ml to about 1.5 mg/ml, or about 200 ng/ml to about 1 mg/ml sonic hedgehog, or about 500 ng/ml sonic hedgehog.
52. The method of any of statements 14-51, wherein the pre-treatment culture medium contains about 1.0 nanomolar to about 2 micromolar, or about 10.0 nanomolar to about 1.0 micromolar, or about 100 nanomolar to about 800 nanomolar, or at about 200 nM to about 700 nM, or at about 500 nanomolar of the WNT signaling activator.
53. The method of any of statements 14-52, wherein the WNT signaling activator is CHIR99021 (6-(2-(4-(2,4-dichlorophenyl)-5-(4-methyl-1H-imidazol-2-yl)pyrimidin-2-ylamino)ethylamino)nicotinonitrile); 1-azakenpaullone (9-Bromo-7,12-dihydro-pyrido[3′,2′:2,3]azepino[4,5-b]indol-6(5H)-one), BIO ((2′Z,3′E)-6-Bromoindirubin-3′-oxime); AR-A014418 (N-(4-Methoxybenzyl)-N′-(5-nitro-1,3-thiazol-2-yl)urea); Indirubin-3′-monoxime; 5-Iodo-indirubin-3′-monoxime; kenpaullone (9-Bromo-7,12-dihydroindolo-[3,2-d] [1]benzazepin-6(5H)-one); SB-415286 (3-[(3-Chloro-4-hydroxyphenyl)amino]-4-(2-nitro-phenyl)-1H-pyrrol-2,5-dione); SB-216763 (3-(2,4-Dichlorophenyl)-4-(1-methyl-1H-indol-3-yl)-1H-pyrrole-2,5-dione); Maybridge SEW00923SC (2-anilino-5-phenyl-1,3,4-oxadiazole); (Z)-5-(2,3-Methylenedioxyphenyl)-imidazolidine-2,4-dione; TWS119 (3-(6-(3-aminophenyI)-7H-pyrrolo[2,3-d]pyrimidin-4-yloxy)phenol); CHIR98014 (N2-(2-(4-(2,4-dichlorophenyl)-5-(1H-imidazol-1 -yl)pyrimidin-2-ylamino)ethyl)-5-nitropyridine-2,6-diamine): SB415286 (3-(3-chloro-4-hydroxyphenylamino)-4-(2-nitrophenyl)-1H-pyrrole-2,5-dione); Tideglusib (2-(1-naphthalenyl)-4-(phenylmethyl)); LY2090314 (3-imidazo[1,2-a]pyridin-3-yl-4-[1,2,3,4-tetrahydro-2-(1-piperidinylcarbonyl)-pyrrolo[3,2,1-jk][1,4]benzodiazepin-7-yl]); lithium salt; or any combination thereof.
54. The method of any of statements 14-53, wherein the ANT signaling activator is CHIR99021.
55. The method of any of statements 14-54, wherein P-zero (P0) and glial fibrillary acidic protein (GFAP) immunoreactivity is not detectable in EPI-NCSC after pre-treatment.
56. The method of any of statements 14-55, wherein the third differentiation culture medium of the cultured cells can be exchanged every 48 hours.
57. The method of any of statements 14-56, furthering comprising administering at least about 100 of the Schwann cells to a mammalian subject.
58. The method of any of statements 14-57, comprising administering at least about 1000, or at least about 10,000, or at least about 100,000, or at least about 1,000,000, or at least about 10,000,000, or at least about 100,000,000 of the Schwann cells to a subject.
59. The method of any of statements 14-58, wherein the Schwann cell(s) is/are allogenic or autologous cell(s) to a subject who may be in need of Schwann cells.
60. The method of any of statements 14-59, wherein the Schwann cell(s) is/are myelinating Schwann cells or precursors of myelinating Schwann cells.
61. The method of any of statements 14-60, further comprising administering the Schwann cells to a subject having, or suspected of having, Guillain-Barre syndrome, multiple sclerosis, Amyotrophic lateral sclerosis (ALS), chronic inflammatory demyelinating polyradiculoneuropathy (CIDP), Experimental allergic encephalomyelitis (EAE), Alzheimer's disease, Parkinson's disease, primary lateral sclerosis, Progressive bulbar palsy, Pseudobulbar palsy, Primary lateral sclerosis (PLS), Progressive muscular atrophy, Spinal muscular atrophy (SMA), including Type I (also called Werdnig-Hoffmann disease), Type II, Type III (Kugelberg-Welander disease). Fazio-Londe disease, Huntington's disease. Kennedy's disease also known as progressive spinobulbar muscular atrophy, hereditary spastic paraplegia (HSP), congenital SMA with arthrogryposis, Post-polio syndrome (PPS), traumatic spinal cord injury, progressive pseudobulbar palsy, progressive muscular atrophy, progressive bulbar palsy, postpolio syndrome, stroke, head trauma, spinal cord injuty, or a combination thereof.
62. A kit comprising:
   a. a first differentiation composition comprising a reducing agent, TGFβ1 inhibitor, and optionally serum;
   b. a second differentiation composition comprising an RARγ agonist, the TGFβ1 inhibitor, and optionally serum;
   c. a third differentiation composition comprising the RARγ agonist, the TGFβ1 inhibitor, a fibroblast growth factor, a platelet derived growth factor, a neuregulin, and an agent that elevates cAMP levels, and optionally serum; and
   d. instructions for generating Schwann cells.
63. The kit of statement 62, wherein each component of the first differentiation composition, the second differentiation composition, and the third differentiation composition is separately packaged.
64. The kit of statement 62, wherein the first differentiation composition, the second differentiation composition, and the third differentiation composition are concentrates for dilution into a culture medium.
65. The kit of statement 62, wherein the serum is packaged separately and the remainder of the first differentiation composition, the second differentiation composition, and the third differentiation composition, are each separately packaged.
66. The kit of statement 62 or 65, wherein the serum is packaged separately and the first differentiation composition, the second differentiation composition, and the third differentiation composition, are each separately packaged as dry compositions.
67. The kit of any of statements 62-66, wherein the serum is fetal bovine serum.
68. The kit of any of statements 62-67, wherein the reducing agent in the first differentiation culture composition is a disulphide bond reducing agent.
69. The kit of any of statements 62-68, wherein the reducing agent is dithiothreitol (DTT), β-mercaptoethanol (βME), or a mixture thereof.
70. The kit of any of statements 62-69, wherein reducing agent β-mercaptoethanol.
71. The kit of any of statements 62-70, wherein the instructions state that the reducing agent is present in the first differentiation culture medium at a concentration of about 0.1 millimolar to about 10 millimolar, or about 0.2 millimolar to about 5 millimolar, or about 0.5 millimolar to about 1.5 millimolar, or about 1 millimolar.
72. The kit of any of statements 62-71, wherein the TGFβ inhibitor is 4-[4-(1,3-benzodioxol-5-yl)-5-(2-pyridinyl)-1H-imidazol-2-yl]benzamide (SB 431542); 3-(6-Methyl-2-pyridinyl)-N-phenyl-4-(4-quinolinyl)-1H-pyrazole-1-carbothioamide (A83-01); 2-(3-(6-Methylpyridine-2-yl)-1H-pyrazol-4-yl)-1,5-naphthyridine (SJN 2511); 4-[4-(2,3-Dihydro-1,4-benzodioxin-6-yl)-5-(2-pyridinyl)1H-imidazol-2-yl]benzamide (D 4476); 4-[3-(2-Pyridinyl)-1H-pyrazol-4-yl]-quinoline (LY 364947); 2-(4-(benzo[d][1,3]dioxol-5-yl)-2-tert-butyl-1H-imidazol-5-yl)-6-methylpyridine (SB505124); 6-[2-(1,1-Dimethylethyl)-5-(6-methyl-2-pyridinyl)-1H-imidazol-4-yl]quinoxaline (SB 525334); 2-(5-Chloro-2-fluorophenyl)-4-[(4-pyridyl)amino]pteridine (SD 2081); 4-(6-(4-(piperazin-1-yl)phenyl)pyrazolo[1,5-a]pyrimidin-3-yl)quinoline (LDN-193189 or any combination thereof.
73. The kit of any of statements 62-72, wherein the TGFβ1 inhibitor is SB431542.
74. The kit of any of statements 62-73, wherein the instructions state that the TGFβ1 inhibitor is at a concentration of about 0.5 micromolar to about 30 micromolar in a solution, or about 1.0 micromolar to about 20 micromolar in a solution, or about 3 micromolar to about 15 micromolar in a solution, or about 10 micromolar in a solution.
75. The kit of any of statements 62-74, wherein the RAR γ agonist is CD1530; CD666; NRX204647; retinoic acid; all-trans retinoic acid (ATRA); 9-cis retinoic acid; all-trans 3-4 didehydro retinoic acid; 4-oxo retinoic acid; Retinol; 4-[(E)-2-(5,6,7,8-tetrahydro-5,5,8,8-tetramethyl-2-naphthalenyl)-1-propenyl]benzoic acid; 4-(5-methoxymethyl-5-methyl-2,3,4,5-tetrahydro-1-benzo[b]oxepin-8-yl-ethynyl)-benzoic acid; 4-(5-ethoxymethyl-5-methyl-2,3,4,5-tetrahydrobenzo[b]oxepin-8-ylethynyl)-benzoic acid; 4-(5-methyl-5-propoxymethyl-2,3,4,5-tetrahydrobenzo[b]oxepin-8-ylethynyl)-benzoic acid; (E)-4-[2-(5-methoxymethyl-5-propyl-2,3,4,5-tetrahydrobenzo[b]oxepin-8-yl-vinyl]-benzoic acid; (E)-4-[2-(5-methoxymethyl-5-methyl-2,3,4,5-tetrahydro-benzo[b]oxepin-8-yl)-vinyl]-benzoic acid; (E)-4-[2-(5-methyl-5-propoxymethyl-2,3,4,5-tetrahydrobenzo[b]oxepin-8-yl)-vinyl]-benzoic acid; 4-(5-methoxymethyl-5-methyl-2,3,4,5-tetrahydrobenzo[b]thiepin-8-ylethynyl)-benzoic acid; 4-(5-ethoxymethyl-5-methyl-2,3,4,5-tetrahydrobenzo[b]thiepin-8-ylethynyl)-benzoic acid; (E)-4-[2-(5-ethoxymethyl-5-methyl-2,3,4,5-tetrahydrobenzo[b]thiepin-8-yl)-vinyl]-benzoic acid; (E)-4-[2-(5-methoxymethyl-5-propyl-2,3,4,5-tetrahydrobenzo[b]thiepin-8-yl)-vinyl]-benzoic acid; 4-(4-methoxymethyl-4-methyl-chroman-6-ylethynyl)-benzoic acid; (E)-4-[2-(4-methoxymethyl-4-methyl-chroman-6-yl)-vinyl]-benzoic acid; or any combination thereof.
76. The kit of any of statements 62-75, wherein the RAR γ agonist is all-trans retinoic acid.
77. The kit of any of statements 62-76, wherein the instructions state that the RARγ agonist is at a concentration of about 5 ng/ml to about 60 ng/ml, or about 20 ng/ml to about 50 ng/ml, or about 35 ng/ml.
78. The kit of any of statements 62-77, wherein the instructions state that the serum concentration in the third differentiation culture medium is less than the serum concentration in the first or second differentiation medium.

79. The kit of any of statements 62-78, wherein the instructions state that the third differentiation culture medium has about 0.1% to about 4%, or about 0.3 to about 3%, or about 0.5% to about 1.5%, or about 1.0% serum.
80. The kit of any of statements 62-79, wherein the RARγ agonist and the TGFγ1 inhibitor are present in the third differentiation culture medium at the same concentrations as in the second differentiation culture media.
81. The kit of any of statements 62-80, wherein the fibroblast growth factor is basic fibroblast growth factor or FGF2.
82. The kit of any of statements 62-81, wherein the instructions state that the fibroblast growth factor is present in the third differentiation culture medium at a concentration of about 1 ng/ml to about 30 ng/ml, or at about 3 ng/ml to about 20 ng/ml, or at about 5 ng/ml to about 15 ng/ml, or about 10 ng/ml.
83. The kit of any of statements 62-82, wherein the platelet derived growth factor (PDGF) is PDGF-B, PDGF-BB, PDGF-AB, PDGF-C, PDGF-D, PDGF-CC, PDGF-DD, PDGF-BC, PDGF-AC, PDGF-AD, PDGF-BD or a combination thereof.
84. The kit of any of statements 62-83, wherein the platelet derived growth factor is PDGF-BB.
85. The kit of any of statements 62-84, wherein the instructions state that the PDGF is present in the third differentiation culture medium at a concentration of about 0.5 ng/ml to about 25 ng/ml, or at about 1 ng/ml to about 15 ng/ml, or at about 2 ng/ml to about 10 ng/ml, or at about 5 ng/ml.
86. The kit of any of statements 62-85, wherein the agent that elevates cAMP levels is forskolin, cholera toxin, caffeine, theophylline, bucladesine (dibutyryl cAMP, db cAMP), pertussis toxin, or a combination thereof.
87. The kit of any of statements 62-86, wherein the instructions state that the adenylyl cyclase is present in the third differentiation culture medium at a concentration of about 0.5 μM to about 25 μM, or at about 1 μM to about 15 μM, or at about 2 μM to about 10 μM, or at about 5 μM.
88. The kit of any of statements 62-87, wherein the instructions state that the neuregulin-1 is present in the third differentiation culture medium at a concentration of about 50 ng/ml to about 500 ng/ml, or at about 100 ng/ml to about 400 ng/ml, or at about 150 ng/ml to about 300 ng/ml, or at about 200 ng/ml.
89. The kit of any of statements 62-88, further comprising a pre-treatment culture medium comprising sonic hedgehog and a WNT signaling activator.
90. The kit of any of statements 62-89, wherein the WNT signaling activator is CHIR99021 (6-(2-(4(2,4-dichlorophenyl)-5-(4-methyl-1H-imidazol-2-yl)pyrimidin-2-ylamino)ethylamino)nicotinonitrile); 1-azakenpaullone (9-Bromo-7,12-dihydro-pyrido[3′,2′:2,3]azepino[4,5-b]indol-6(5H)-one), BIO ((2′Z,3′E)-6-Bromoindirubin-3′-oxime); AR-A014418 (N-(4-Methoxybenzyl)-N′-(5-nitro-1,3-thiazol-2-yl)urea); Indirubin-3′-monoxime; 5-Iodo-indirubin-3′-monoxime; kenpaullone (9-Bromo-7,12-dihydroindolo-[3,2-d] [1]benzazepin-6(5H)-one); SB-415286 (3-[(3-Chloro-4-hydroxyphenyl)amino]-4-(2-nitro-phenyl)-1H-pyrrole-2,5-dione); SB-216763 (3-(2,4-Dichlorophenyl)-4-(1-methyl-1H-indol-3-yl)-1H-pyrrole-2,5-dione); Maybridge SEW00923SC (2-anilino-5-phenyl-1,3,4-oxadiazole); (Z)-5-(2,3-MethylenedioxyphenyI)-imidazolidine-2,4-dione; TWS119 (3-(6-(3-aminophenyI)-7H-pyrrolo[2,3-d]pyrimidin-4-yloxy)phenol); CHIR98014 (N2-(2-(4-(2,4-dichlorophenyl)-5-(1H-imidazol-1-yl)pyrimidin-2-ylamino)ethyl)-5-nitropyridine-2,6-diamine); SB415286 (3-(3-chloro-4-hydroxyphenylamino)-4-(2-nitrophenyl)-1H-pyrrole-2,5-dione); Tideglusib (2-(1-naphthalenyl)-4-(phenylmethyl)); LY2090314 (3-imidazo[1,2-a]pyridin-3-yl-4-[1,2,3,4-tetrahydro-2-(1-piperidinylcarbonyI)-pyrrolo[3,2,1-jk][1,4]benzodiazepin-7-yl]); lithium salt; or any combination thereof.
91. The kit of any of statements 62-90, wherein WNT signaling activator is CHIR99021.
92. The kit of any of statements 62-91, further comprising components for in vitro cell culture of epidermal neural crest stem cells, partially differentiated Schwann cells, or Schwann cells, or components for culture of epidermal neural crest stem cells, partially differentiated Schwann cells, and Schwann cells.
93. The kit of any of statements 62-92, further comprising one or more cell collection device for collecting epidermal neural crest stem cells.
94. The kit of any of statements 62-93, further comprising at least one cell culture medium.
95. The kit of any of statements 62-94, further comprising a population of epidermal neural crest stein cells.
96. The kit of any of statements 62-95, further comprising a diluent, a pharmaceutically acceptable carrier, a syringe, a catheter, or a device for delivery of cells or of the composition.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a compound,” “a cell,” “a component” or “a compound” includes a plurality of such compounds, cells, components, or compounds (for example, a solution of cells, components, or compounds; a suspension of cells, or a series of components or compounds; or a series of cell preparations), and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. A method of generating Schwann cells comprising:
a. contacting one or more epidermal neural crest stem cells (EPI-NCSC) with a first differentiation culture medium comprising β-mercaptoethanol, SB431542, and a first amount of fetal bovine serum for 1-3 days to generate a first cell population;
b. contacting the first cell population with a second differentiation culture medium comprising all-trans-retinoic acid, SB431542, and the first amount of serum for 1-4 days to generate a second cell population; and
c. contacting the second cell population with a third differentiation culture medium comprising all-trans-retinoic acid, SB431542, fetal bovine serum, fibroblast growth factor-2, a platelet derived growth factor-BB, neuregulin-1, and forskolin for about 4 to about 28 days to thereby generate EPI-NCSC-derived Schwann cells.

2. The method of claim 1, wherein the EPI-NCSC-derived Schwann cells express KROX20, SOX10, P-zero (P0), MBP, or a combination thereof.

3. The method of claim 1, wherein the EPI-NCSC-derived Schwann cells express SOX10, KROX20, or a combination thereof in cell nuclei.

4. The method of claim 1, wherein the β-mercaptoethanol is present in the first differentiation culture medium at a concentration of about 1 millimolar.

5. The method of claim 1, wherein the SB431542 is present in the first, second, and third differentiation culture medium at a concentration sufficient to inhibit neuron formation.

6. The method of claim 1, wherein the SB431542 is present in the first, second, and third differentiation culture medium at a concentration of about 3 micromolar to about 15 micromolar.

7. The method of claim 1, wherein the first amount of fetal bovine serum in the first and second differentiation medium is about 5% to about 15% fetal bovine serum.

8. The method of claim 1, wherein the second amount of fetal bovine serum in the third differentiation medium is about 0.2% to about 2% fetal bovine serum.

9. The method of claim 1, wherein the all-trans-retinoic acid is present in the second and third differentiation culture medium at a concentration of about 10 ng/ml to about 60 ng/ml.

10. The method of claim 1, wherein the all-trans-retinoic acid is present in the third differentiation culture medium for more than 5 days.

11. The method of claim 1, wherein the fibroblast growth factor-2 in the third differentiation culture medium is at a concentration of about 2 ng/ml to about 20 ng/ml.

12. The method of claim 1, wherein the recombinant human PDGF-BB in the third differentiation culture medium is at a concentration of about 1 ng/ml to about 15 ng/ml.

13. The method of claim 1, wherein the forskolin in the third differentiation culture medium is at a concentration of about 1 μM to about 20 μM.

14. The method of claim 1, wherein the neuregulin-1 in the third differentiation culture medium is at a concentration of about 50 ng/ml to about 500 ng/ml.

15. The method of claim 1, further comprising pre-treating the epidermal neural crest stem cells (EPI-NCSC) before contacting them with the first differentiation culture medium, comprising:
a. contacting one or more epidermal neural crest stem cells (EPI-NCSC) with a pre-treatment culture medium comprising sonic hedgehog and CHIR99021 for at least 1 day to generate pre-treated epidermal neural crest stem cells; and
b. incubating one or more of the pre-treated epidermal neural crest stem cells in a culture media without sonic hedgehog and CHIR99021 for at least one day to thereby generate one or more pre-treated epidermal neural crest stem cells.

16. The method of claim 15, further comprising obtaining the epidermal neural crest stem cells from one or more hair follicle bulge.

17. The method of claim 15, wherein the sonic hedgehog and/or the CHIR99021 are present in the pre-treatment culture medium at a concentration sufficient to increase nuclear SOX10 expression.

18. The method of claim 15, wherein the pre-treatment culture medium contains about 200 ng/ml to about 1 mg/ml sonic hedgehog.

19. The method of claim 15, wherein the pre-treatment culture medium contains about 200 nM to about 700 nM of the CHIR99021.

20. The method of claim 15, wherein the pre-treatment culture medium further comprises FGF2, EGF, L-alanyl-L-glutamine dipeptide, one or more antibiotic, one or more anti-fungal, or a combination thereof.

21. The method of claim 1, wherein the epidermal neural crest stem cells are mammalian epidermal neural crest stem cells.

22. The method of claim 1, wherein the Schwann cells are myelinating Schwann cells or precursors of myelinating Schwann cells.
